(19) United States
(12) Patent Application Publication
(10) Pub. No.: US 2009/0137554 A1
(43) Pub. Date: May 28, 2009

(54) 1,4,5,6-TETRAHYDRO -PYRROLO[2,3-d]AZEPINES AND -IMIDAZO[4,5-d]AZEPINES AS MODULATORS OF NUCLEAR RECEPTOR ACTIVITY

(75) Inventors: John Francis Mehlmann, King of Prussia, PA (US); Joseph Theodore Lundquist, IV, Limerick, PA (US); Paige Erin Mahaney, Pottstown, PA (US); Matthew Lantz Crawley, Phoenixville, PA (US); Callain Younghee Kim, Collegeville, PA (US)

Correspondence Address: WYETH/FINNEGAN HENDERSON, LLP, 901 NEW YORK AVENUE, NW, WASHINGTON, DC, 20001-4413, US

(73) Assignee: Wyeth, Madison, NJ (US)

(21) Appl. No.: 12/255,216

(22) Filed: Oct. 21, 2008

Related U.S. Application Data
(60) Provisional application No. 60/999,990, filed on Oct. 22, 2007.

(51) Int. Cl. A61K 31/55; C07D 487/04; C07D 491/20; A61P 3/04

(52) U.S. Cl. 514/212.02; 540/580; 514/215; 540/543

(57) ABSTRACT

Disclosed are chemical entities including compounds of Formula I

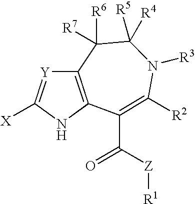

and pharmaceutically acceptable salts thereof, wherein X is chosen from CN, CF₃, CF₂H, S(O)ₙR⁸, and S(O)₂N(R⁹)R¹⁰; Y is chosen from CR¹¹ and N; Z is chosen from O and NH; R³ is chosen from —C(O)R¹² and —C(O)N(R⁹)R¹⁰; and n, R¹, R² and R⁴-R¹² are defined herein; compositions comprising one or more such chemical entities; and methods of using one or more such chemical entities for modulating the activity of certain receptors (e.g., farnesoid X) or for the treatment or prevention of one or more symptoms of disease or disorder related to the activity of those receptors.

This application claims the benefit of U.S. provisional application No. 60/999,990, filed Oct. 22, 2007, the disclosure of which is incorporated herein by reference in its entirety.

Certain chemical entities, compositions and methods are provided for modulating the activity of certain receptors or for the treatment or prevention of one or more symptoms of disease or disorder related to the activity of those receptors.

Nuclear receptors are a superfamily of regulatory proteins that are structurally and functionally related and are receptors for, e.g., steroids, retinoids, vitamin D and thyroid hormones (see, e.g., Evans (1988) Science 240:889-895). These proteins bind to cis-acting elements in the promoters of their target genes and modulate gene expression in response to ligands for the receptors.

Nuclear receptors can be classified based on their DNA binding properties (see, e.g., Evans, supra and Glass (1994) Endocr. Rev. 15:391-407). For example, one class of nuclear receptors includes the glucocorticoid, estrogen, androgen, progestin and mineralocorticoid receptors which bind as homodimers to hormone response elements (HREs) organized as inverted repeats (see, e.g., Glass, supra). A second class of receptors, including those activated by retinoic acid, thyroid hormone, vitamin D₃, fatty acids/peroxisome proliferators (i.e., peroxisome proliferator activated receptor (PPAR)) and ecdysone, bind to HREs as heterodimers with a common partner, the retinoid X receptors (i.e., RXRs, also known as the 9-cis retinoic acid receptors; see, e.g., Levin et al. (1992) Nature 355:359-361 and Heyman et al. (1992) Cell 68:397-406).

RXRs are unique among the nuclear receptors in that they bind DNA as a homodimer and are required as a heterodimeric partner for a number of additional nuclear receptors to bind DNA (see, e.g., Mangelsdorf et al. (1995) Cell 83:841-850). The latter receptors, termed the class II nuclear receptor subfamily, include many which are established or implicated as important regulators of gene expression. There are three RXR genes (see, e.g., Mangelsdorf et al. (1992) Genes Dev. 6:329-344), coding for RXRα, -β, and -γ, all of which are able to heterodimerize with any of the class II receptors, although there appear to be preferences for distinct RXR subtypes by partner receptors in vivo (see, e.g., Chiba et al. (1997) Mol. Cell. Biol. 17:3013-3020). In the adult liver, RXRα is the most abundant of the three RXRs (see, e.g., Mangelsdorf et al. (1992) Genes Dev. 6:329-344), suggesting that it might have a prominent role in hepatic functions that involve regulation by class II nuclear receptors. See also, Wan et al. (2000) Mol. Cell. Biol 20:4436-4444.

Included in the nuclear receptor superfamily of regulatory proteins are nuclear receptors for which the ligand is known and those which lack known ligands. Nuclear receptors falling in the latter category are referred to as orphan nuclear receptors. The search for activators for orphan receptors has led to the discovery of previously unknown signaling pathways (see, e.g., Levin et al., (1992), supra and Heyman et al., (1992), supra). For example, it has been reported that bile acids, which are involved in physiological processes such as cholesterol catabolism, are ligands for the farnesoid X receptor (infra).

Since it is known that products of intermediary metabolism act as transcriptional regulators in bacteria and yeast, such molecules may serve similar functions in higher organisms (see, e.g., Tomkins (1975) Science 189:760-763 and O'Malley (1989) Endocrinology 125:1119-1120). For example, one biosynthetic pathway in higher eukaryotes is the mevalonate pathway, which leads to the synthesis of cholesterol, bile acids, porphyrin, dolichol, ubiquinone, carotenoids, retinoids, vitamin D, steroid hormones and farnesylated proteins.

The farnesoid X receptor (originally isolated as RIP14 (retinoid X receptor-interacting protein-14), see, e.g., Seol et al. (1995) Mol. Endocrinol. 9:72-85) is a member of the nuclear hormone receptor superfamily and is primarily expressed in the liver, kidney and intestine (see, e.g., Seol et al., supra and Forman et al. (1995) Cell 81:687-693). It functions as a heterodimer with the retinoid X receptor (RXR) and binds to response elements in the promoters of target genes to regulate gene transcription. The farnesoid X receptor-RXR heterodimer binds with highest affinity to an inverted repeat-1 (IR-1) response element, in which consensus receptor-binding hexamers are separated by one nucleotide. The farnesoid X receptor is part of an interrelated process, in that the receptor is activated by bile acids (the end product of cholesterol metabolism) (see, e.g., Makishima et al. (1999) Science 284:1362-1365, Parks et al. (1999) Science 284:1365-1368, Wang et al. (1999) Mol. Cell. 3:543-553), which serve to inhibit cholesterol catabolism. See also, Urizar et al. (2000) J. Biol. Chem. 275:39313-39317.

Nuclear receptor activity, including the farnesoid X receptor and/or orphan nuclear receptor activity, has been implicated in a variety of diseases and disorders, including, but not limited to, hyperlipidemia and hypercholesterolemia, and complications thereof, including without limitation coronary artery disease, angina pectoris, carotid artery disease, strokes, cerebral arteriosclerosis and xanthoma, (see, e.g., International Patent Application Publication No. WO 00/57915), hyperlipoproteinemia (see, e.g., International Patent Application Publication No. WO 01/60818), hypertriglyceridemia, lipodystrophy, peripheral occlusive disease, ischemic stroke, hyperglycemia and diabetes mellitus (see, e.g., International Patent Application Publication No. WO 01/82917), disorders related to insulin resistance including the cluster of disease states, conditions or disorders that make up “Syndrome X” such as glucose intolerance, an increase in plasma triglyceride and a decrease in high-density lipoprotein cholesterol concentrations, hypertension, hyperuricemia, smaller denser low-density lipoprotein particles, and higher circulating levels of plasminogen activator inhibitor-1, atherosclerosis and gallstones (see, e.g., International Patent Application Publication No. WO 00/37077), disorders of the skin and mucous membranes (see, e.g., U.S. Pat. Nos. 6,184,215 and 6,187,814, and International Patent Application Publication No. WO 98/32444), obesity, acne (see, e.g., International Patent Application Publication No. WO 00/49992), and cancer, cholestasis, Parkinson's disease and Alzheimer's disease (see, e.g., International Patent Application Publication No. WO 00/17334).

The activity of nuclear receptors, including the farnesoid X receptor and/or orphan nuclear receptors, has been implicated in physiological processes including, but not limited to, triglyceride metabolism, catabolism, transport or absorption, bile acid metabolism, catabolism, transport, absorption, re-absorption or bile pool composition, cholesterol metabolism, catabolism, transport, absorption, or re-absorption. The modulation of cholesterol 7α-hydroxylase gene (CYP7A1) transcription (see, e.g., Chiang et al. (2000) J. Biol. Chem. 275:10918-10924), HDL metabolism (see, e.g., Urizar et al. (2000) J. Biol. Chem. 275:39313-39317), hyperlipidemia, cholestasis, and increased cholesterol efflux and increased expression of ATP binding cassette transporter protein (ABC1) (see, e.g., International Patent Application Publication No. WO 00/78972) are also modulated or otherwise affected by the farnesoid X receptor.

Thus, there is a need for compounds, compositions and methods of modulating the activity of nuclear receptors, including the farnesoid X receptor and/or orphan nuclear receptors. Such compounds may be useful in the treatment or prevention of one or more symptoms of diseases or disorders in which nuclear receptor activity is implicated.

Provided is at least one chemical entity chosen from compounds of Formula I

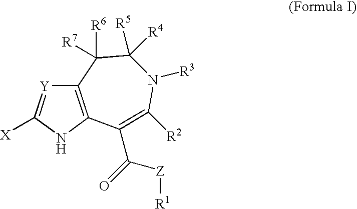

and pharmaceutically acceptable salts thereof, wherein
X is chosen from CN, CF₃, CF₂H, S(O)ₙR⁸, and S(O)₂N(R⁹)R¹⁰;
n is 0, 1, or 2;
Y is chosen from CR¹¹ and N;
Z is chosen from O and NH;
R¹ is chosen from optionally substituted alkyl, optionally substituted cycloalkyl, optionally substituted heterocyclyl, optionally substituted aryl, and optionally substituted heteroaryl;
R² is chosen from hydrogen and optionally substituted alkyl;
R³ is chosen from —C(O)R¹² and —C(O)N(R⁹)R¹⁰;
R⁴, R⁵, R⁶ and R⁷ are independently chosen from hydrogen and optionally substituted alkyl, or any two of R⁴, R⁵, R⁶ and R⁷, together with the atoms to which they are attached, form an optionally substituted cycloalkyl or optionally substituted heterocyclyl ring;
R⁸ is chosen from optionally substituted alkyl, optionally substituted cycloalkyl, optionally substituted heterocyclyl, optionally substituted aryl, and optionally substituted heteroaryl;
each occurrence of R⁹ and R¹⁰ is independently chosen from hydrogen, optionally substituted aryl, optionally substituted heteroaryl, optionally substituted alkyl, optionally substituted cycloalkyl, and optionally substituted heterocyclyl, or R⁹ and R¹⁰, together with the atoms to which they are attached, form an optionally substituted heterocyclyl ring;
R¹¹ is chosen from hydrogen and lower alkyl; and
R¹² is chosen from hydrogen, optionally substituted aryl, optionally substituted heteroaryl, optionally substituted alkyl, optionally substituted cycloalkyl, and optionally substituted heterocyclyl.

Also provided is a pharmaceutical composition comprising a pharmaceutically acceptable carrier or excipient and at least one chemical entity described herein.

Also provided is a method of treating or preventing one or more symptoms of a disease or disorder in which nuclear receptor activity is implicated, comprising administering to a subject in need thereof an effective amount of at least one chemical entity described herein.

Also provided is a method of reducing plasma cholesterol levels, in a subject in need thereof, comprising administering an effective amount of at least one chemical entity described herein.

Also provided is a method of reducing plasma triglyceride levels in a subject in need thereof, comprising administering an effective amount of at least one chemical entity described herein.

Also provided is a method of treating or preventing one or more symptoms of a disease or disorder which is affected by abnormal cholesterol, triglyceride, or bile acid levels, comprising administering to a subject in need thereof an effective amount of at least one chemical entity described herein.

Also provided is a method of modulating cholesterol metabolism, catabolism, synthesis, absorption, re-absorption, secretion or excretion in a mammal, comprising administering an effective amount of at least one chemical entity described herein.

Also provided is a method for modulating farnesoid X receptor activity comprising contacting a cell with at least one chemical entity described herein.

Unless defined otherwise, technical and scientific terms used herein have the same meaning as is commonly understood by one of ordinary skill in the art to which this invention belongs. In the event that there are a plurality of definitions for a term herein, those in this section prevail unless stated otherwise.

As used herein, a nuclear receptor is a member of a superfamily of regulatory proteins that are receptors for, e.g., steroids, retinoids, vitamin D and thyroid hormones. These proteins bind to cis-acting elements in the promoters of their target genes and modulate gene expression in response to a ligand therefor. Nuclear receptors may be classified based on their DNA binding properties. For example, the glucocorticoid, estrogen, androgen, progestin and mineralocorticoid receptors bind as homodimers to hormone response elements (HREs) organized as inverted repeats. Another example are receptors, including those activated by retinoic acid, thyroid hormone, vitamin D₃, fatty acids/peroxisome proliferators and ecdysone, that bind to HREs as heterodimers with a common partner, the retinoid X receptor (RXR). Among the latter receptors is the farnesoid X receptor.

As used herein, an orphan nuclear receptor is a gene product that embodies the structural features of a nuclear receptor that was identified without any prior knowledge of their association with a putative ligand and/or for which the natural ligand is unknown. Under this definition, orphan nuclear receptors include, without limitation, farnesoid X receptors, liver X receptors (LXR α & β), retinoid X receptors (RXR α, β & γ), and peroxisome proliferator activator receptors (PPAR α, β & γ) (see, Giguere, Endocrine Reviews (1999), Vol. 20, No. 5: pp. 689-725).

As used herein, farnesoid X receptor refers to all mammalian forms of such receptor including, for example, alternative splice isoforms and naturally occurring isoforms (see, e.g. Huber et al, Gene (2002), Vol. 290, pp.:35-43). Representative farnesoid X receptor species include, without limitation the rat (GenBank Accession No. NM_{—}021745), mouse (Genbank Accession No. NM_{—}009108), and human (GenBank Accession No. NM_{—}005123) forms of the receptor.

As used herein, treatment means any manner in which one or more of the symptoms of a disease or disorder are ameliorated or otherwise beneficially altered. Treatment also encompasses any pharmaceutical use of the compositions herein, such as use for treating nuclear receptor mediated diseases or disorders, or diseases or disorders in which nuclear receptor activity, including the farnesoid X receptor or orphan nuclear receptor activity, is implicated.

As used herein, amelioration of the symptoms of a particular disorder by administration of a particular compound or pharmaceutical composition refers to any lessening, whether permanent or temporary, lasting or transient that can be attributed to or associated with administration of the composition.

As used herein, IC₅₀ refers to an amount, concentration or dosage of a particular test compound that achieves a 50% inhibition of a maximal response, such as modulation of nuclear receptor, including the farnesoid X receptor, activity, in an assay that measures such response.

As used herein, EC₅₀ refers to a dosage, concentration or amount of a particular test compound that elicits a dose-dependent response at 50% of maximal expression of a particular response that is induced, provoked or potentiated by the particular test compound.

It is to be understood that the compounds provided herein may contain chiral centers. Such chiral centers may be of either the (R) or (S) configuration, or may be a mixture thereof. Thus, the compounds of Formula I may be enantiomerically pure, or be stereoisomeric or diastereomeric mixtures. Optically active (+) and (−), (R)- and (S)-, or (D)- and (L)-isomers may be prepared using chiral synthons or chiral reagents, or resolved using conventional techniques, such as HPLC. In those situations, the single enantiomers or diastereomers, i.e., optically active forms, can be obtained by asymmetric synthesis or by resolution of the racemates. Resolution of the racemates can be accomplished, for example, by conventional methods such as crystallization in the presence of a resolving agent, or chromatography, using, for example a chiral high-performance liquid chromatography (HPLC) column.

When the compounds described herein contain olefinic double bonds or other centers of geometric asymmetry, and unless specified otherwise, it is intended that the compounds include both E and Z geometric isomers. Likewise, all tautomeric forms are also intended to be included.

Compounds of Formula I also include crystal forms including polymorphs and clathrates.

Acids (and bases) which are generally considered suitable for the formation of pharmaceutically acceptable salts from basic (or acidic) pharmaceutical compounds are discussed, for example, by S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; in The Orange Book (Food & Drug Administration, Washington, D.C. on their website); and P. Heinrich Stahl, Camille G. Wermuth (Eds.), Handbook of Pharmaceutical Salts: Properties, Selection, and Use, (2002) Int'l. Union of Pure and Applied Chemistry, pp. 330-331. These disclosures are incorporated herein by reference thereto.

Depending on its structure, the phrase “pharmaceutically acceptable salt,” as used herein, refers to a pharmaceutically acceptable organic or inorganic acid or base salt. Representative pharmaceutically acceptable salts include, e.g., alkali metal salts, alkali earth salts, ammonium salts, water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts. Furthermore, a pharmaceutically acceptable salt can have more than one charged atom in its structure. In this instance the pharmaceutically acceptable salt can have multiple counterions. Hence, a pharmaceutically acceptable salt can have one or more charged atoms and/or one or more counterions.

Further, pharmaceutically acceptable salts include, but are not limited to aluminium, ammonium, calcium, copper, ferric, ferrous, lithium, magnesium, manganic, manganous, potassium, sodium, and zinc salts as well as salts derived from pharmaceutically acceptable organic non-toxic bases, such as salts of primary, secondary, and tertiary amines, substituted amines including naturally occurring substituted amines, cyclic amines, and basic ion exchange resins, e.g., arginine, betaine, caffeine, chloroprocaine, choline, N,N′-dibenzylethylenediamine (benzathine), dicyclohexylamine, diethanolamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, iso-propylamine, lidocaine, lysine, meglumine, N-methyl-D-glucamine, morpholine, piperazine, piperidine, polyamine resins, procaine, purines, theobromine, triethanolamine, triethylamine, trimethylamine, tripropylamine, and tris-(hydroxymethyl)-methylamine (tromethamine).

In addition, if the compound of Formula I is obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare non-toxic pharmaceutically acceptable addition salts.

Compounds of Formula I also include prodrugs, for example ester or amide derivatives of the compounds of Formula I. As used herein, a prodrug is a compound that, upon in vivo administration, is metabolized by one or more steps or processes or otherwise converted to the biologically, pharmaceutically or therapeutically active form of the compound. To produce a prodrug, the pharmaceutically active compound is modified such that the active compound will be regenerated by metabolic processes. The prodrug may be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

The term “prodrugs”, as the term is used herein, is also intended to include any covalently bonded carriers which release an active parent drug in vivo when such prodrug is administered to a patient. Since prodrugs are known to enhance numerous desirable qualities of pharmaceuticals (i.e., solubility, bioavailability, manufacturing, etc.) the chemical entities described herein may be delivered in prodrug form. Thus, the skilled artisan will appreciate that the chemical entities described herein encompasses prodrugs, methods of delivering the same, and compositions containing the same. Prodrugs may be prepared by modifying functional groups present in the compound in such a way that the modifications are cleaved, either in routine manipulation or in vivo, to form the parent compound. The transformation in vivo may be, for example, as the result of some metabolic process, such as chemical or enzymatic hydrolysis of a carboxylic, phosphoric or sulphate ester, or reduction or oxidation of a susceptible functionality. Prodrugs include chemical entities wherein a hydroxy, amino, or sulfhydryl group is bonded to any group that, when the prodrug is administered to a patient, it cleaves to form a free hydroxyl, free amino, or free sulfydryl group, respectively. Functional groups which may be rapidly transformed, by metabolic cleavage, in vivo form a class of groups reactive with the carboxyl group of the chemical entities described herein. They include, but are not limited to such groups as alkanoyl (such as acetyl, propionyl, butyryl, and the like), unsubstituted and substituted aroyl (such as benzoyl and substituted benzoyl), alkoxycarbonyl (such as ethoxycarbonyl), trialkylsilyl (such as trimethyl- and triethysilyl), monoesters formed with dicarboxylic acids (such as succinyl), and the like. Because of the ease with which the metabolically cleavable groups of the chemical entities described herein are cleaved in vivo, the compounds bearing such groups can act as prodrugs. The compounds bearing the metabolically cleavable groups have the advantage that they may exhibit improved bioavailability as a result of enhanced solubility and/or rate of absorption conferred upon the parent compound by virtue of the presence of the metabolically cleavable group. A thorough discussion of prodrugs is provided in the following: Design of Prodrugs, H. Bundgaard, ed., Elsevier, 1985; Methods in Enzymology, K. Widder et al, Ed., Academic Press, 42, p. 309 396, 1985; A Textbook of Drug Design and Development, Krogsgaard-Larsen and H. Bundgaard, ed., Chapter 5; “Design and Applications of Prodrugs” p. 113 191, 1991; Advanced Drug Delivery Reviews, H. Bundgard, 8, p. 1 38, 1992; Journal of Pharmaceutical Sciences, 77, p. 285, 1988; Chem. Pharm. Bull., N. Nakeya et al, 32, p. 692, 1984; Pro-drugs as Novel Delivery Systems, T. Higuchi and V. Stella, Vol. 14 of the A.C.S. Symposium Series, and Bioreversible Carriers in Drug Design, Edward B. Roche, ed., American Pharmaceutical Association and Pergamon Press, 1987; Bundgaard, H., Advanced Drug Delivery Review, 1992, 8, 1 38.

The term “solvate” refers to a substance formed by the interaction of a solvent and a compound. Suitable solvates are pharmaceutically acceptable solvates, such as hydrates, including monohydrates and hemi-hydrates. The term “solvate” is intended to include solvates of compounds of Formula I. Similarly, “salts” includes solvates of salts of compounds of Formula I.

The term “chelate” refers to a substance formed by the coordination of a compound to a metal ion at two (or more) points. The term “compound” is intended to include chelates of compounds of Formula I. Similarly, “salts” includes chelates of salts of compounds of Formula I.

The term “non-covalent complex” refers to a substance formed by the interaction of a compound and another molecule wherein a covalent bond is not formed between the compound and the molecule. For example, complexation can occur through van der Waals interactions, hydrogen bonding, and electrostatic interactions (also called ionic bonding). The term “compound” is intended to include non-covalent complexes of compounds of Formula I. Similarly, “salts” includes non-covalent complexes of salts of compounds of Formula I.

The term “hydrogen bond” refers to a form of association between an electronegative atom (also known as a hydrogen bond acceptor) and a hydrogen atom attached to a second, relatively electronegative atom (also known as a hydrogen bond donor). Suitable hydrogen bond donor and acceptors are well understood in medicinal chemistry (G. C. Pimentel and A. L. McClellan, The Hydrogen Bond, Freeman, San Francisco, 1960; R. Taylor and O. Kennard, “Hydrogen Bond Geometry in Organic Crystals”, Accounts of Chemical Research, 17, pp. 320-326 (1984)).

As used herein the terms “group”, “radical” or “fragment” are synonymous and are intended to indicate functional groups or fragments of molecules attachable to a bond or other fragments of molecules.

As used herein, substantially pure means sufficiently homogeneous to appear free of readily detectable impurities as determined by standard methods of analysis, such as thin layer chromatography (TLC), gel electrophoresis, high performance liquid chromatography (HPLC) and mass spectrometry (MS), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound may, however, be a mixture of stereoisomers. In such instances, further purification might increase the specific activity of the compound.

As used herein, “alkyl”, “alkenyl” and “alkynyl” are straight or branched hydrocarbon chains, and if not specified, contain from 1 to 20 carbons or 2 to 20 carbons, such as from 1 to 16 carbons or 2 to 16 carbons. Alkenyl carbon chains having 2 to 20 carbons, in certain embodiments, contain 1 to 8 double bonds and alkenyl carbon chains having 2 to 16 carbons, in certain embodiments, contain 1 to 5 double bonds. Alkynyl carbon chains having 2 to 20 carbons, in certain embodiments, contain 1 to 8 triple bonds, and the alkynyl carbon chains having 2 to 16 carbons, in certain embodiments, contain 1 to 5 triple bonds. Alkyl, alkenyl and alkynyl groups may be optionally substituted as described herein. Exemplary alkyl, alkenyl and alkynyl groups herein include, but are not limited to, methyl, ethyl, propyl, isopropyl, isobutyl, n-butyl, sec-butyl, tert-butyl, isopentyl, neopentyl, tert-pentyl, isohexyl, allyl (propenyl) and propargyl (propynyl). As used herein, lower alkyl, lower alkenyl, and lower alkynyl refer to carbon chains having from 1 to 6 carbons (lower alkyl) or from 2 to 6 carbons (lower alkenyl and lower alkynyl).

As used herein, “alkylene” refers to a straight or branched divalent aliphatic hydrocarbon group wherein the alkylene is attached to the rest of the molecule through two different bonds in the alkylene. In some embodiments the alkylene has from 1 to 20 carbon atoms, in another embodiment the alkylene has from 1 to 12 carbons. Alkylene groups may be optionally substituted as described herein. The term “lower alkylene” refers to alkylene groups having 1 to 6 carbons. In certain embodiments, alkylene groups are lower alkylene, including alkylene of 1 to 3 carbon atoms.

As used herein, “alkoxy” refers to an alkyl group attached through an oxygen bridge such as, for example, methoxy, ethoxy, propoxy, isopropoxy, n-butoxy, sec-butoxy, tert-butoxy, pentoxy, 2-pentyloxy, isopentoxy, neopentoxy, hexoxy, 2-hexoxy, 3-hexoxy, 3-methylpentoxy, and the like. In some embodiments, alkoxy groups have from 1 to 6 carbon atoms attached through the oxygen bridge. The alkyl portion of alkoxy groups may be optionally substituted as described herein. “Lower alkoxy” refers to alkoxy groups having 1 to 6 (e.g., 1 to 4) carbons.

As used herein, “aralkyl” refers to a radical of the formula —R^{a}R^{d} where R^{a} is an alkyl radical as defined above, substituted by R^{d}, an aryl radical, as defined herein, e.g., benzyl. The alkyl and aryl radicals independently may be optionally substituted as described herein.

As used herein, “aryl” refers to aromatic monocyclic or multicyclic ring system containing from 6 to 14 carbon atoms. Aryl groups include, but are not limited to groups such as unsubstituted or substituted phenyl and unsubstituted or substituted naphthyl. Aryl groups may be optionally substituted as described herein.

As used herein, “cycloalkyl” refers to a saturated mono- or multi-cyclic ring system, in certain embodiments of 3 to 10 carbon atoms, in other embodiments of 3 to 6 carbon atoms. Cycloalkyl groups include multicyclic ring systems containing from 7 to 14 carbon atoms, where at least one ring is aromatic and at least one ring is partially or fully saturated (e.g., unsubstituted or substituted fluorenyl). Cycloalkyl groups also include mono- or multicyclic ring systems that respectively include at least one double bond or at least one triple bond (i.e., cycloalkenyl and cycloalkynyl). Cycloalkenyl groups may contain 3 to 10 carbon atoms, or 4 to 7 carbon atoms. Cycloalkynyl groups may contain 3 to 10 carbon atoms, or 8 to 10 carbon atoms. The ring systems of the cycloalkyl groups may be composed of one ring or two or more rings which may be joined together in a fused, bridged or spiro-connected fashion. Cycloalkyl groups may be optionally substituted as described herein.

As used herein, “cycloalkylalkyl” refers to a radical of the formula —R^{a}R^{b} where R^{a} is an alkyl radical as defined above and R^{b} is a cycloalkyl radical as defined above. The alkyl radical and the cycloalkyl radical independently may be optionally substituted as defined above.

As used herein, “heteroaralkyl” refers to a radical of the formula —R^{a}R^{e} where R^{a} is an alkyl radical as defined above and R^{e} is a heteroaryl radical as defined herein. The alkyl radical and the heterocyclyl radical independently may be optionally substituted as defined herein.

As used herein, “heteroaryl” refers to a monocyclic or multicyclic aromatic heterocyclyl, as defined herein, in certain embodiments, of 5 to 15 members where one or more, (e.g., 1 to 3) of the atoms in the ring system is a heteroatom selected from nitrogen, oxygen and sulfur. The heteroaryl group may be optionally fused to a benzene ring. Heteroaryl groups may be optionally substituted as defined herein. Heteroaryl groups include, but are not limited to, furyl, imidazolyl, pyrimidinyl, tetrazolyl, thienyl, pyridyl, pyrrolyl, thiazolyl, isothiazolyl, oxazolyl, isoxazolyl, triazolyl, acridinyl, benzimidazolyl, benzothiazolyl, benzindolyl, benzothiadiazolyl, benzonaphthofuranyl, benzoxazolyl, benzofuranyl, benzothiophenyl, benzotriazolyl, benzo[4,6]imidazo[1,2-a]pyridinyl, carbazolyl, cinnolinyl, dibenzofuranyl, indolyl, indazolyl, isoindolyl, indolizinyl, naphthyridinyl, phenazinyl, phenothiazinyl, phenoxazinyl, phthalazinyl, pteridinyl, purinyl, quinazolinyl, quinoxalinyl, quinolinyl, and isoquinolinyl.

As used herein, “heterocyclyl” refers to a stable 3- to 18-membered ring system which consists of carbon atoms and from one to five heteroatoms selected from the group consisting of nitrogen, oxygen and sulfur. The heterocyclyl radical may be a monocyclic, bicyclic, tricyclic or tetracyclic ring system, which may include fused or bridged ring systems; the nitrogen, carbon or sulfur atoms in the heterocyclyl radical may be optionally oxidized; the nitrogen atom may be optionally quaternized; and the ring radical may be partially or fully saturated. Heterocyclyl groups may be optionally substituted as defined herein. Examples of such heterocyclyl radicals include, but are not limited to, dioxolanyl, decahydroisoquinolyl, furanonyl. imidazolinyl, imidazolidinyl, isothiazolidinyl, indolinyl, isoindolinyl, isoxazolidinyl, morpholinyl, octahydroindolyl, octahydroisoindolyl, 2-oxopiperazinyl, 2-oxopiperidinyl, 2-oxopyrrolidinyl, 2-oxoazepinyl, oxazolidinyl, oxiranyl, piperidinyl, piperazinyl, 4-piperidonyl, pyrrolidinyl, pyrazolidinyl, quinuclidinyl, thiazolidinyl, tetrahydrofuryl, tetrahydropyranyl, thiamorpholinyl, thiamorpholinyl sulfoxide, and thiamorpholinyl sulfone.

As used herein, “heterocyclylalkyl” refers to a radical of the formula —R^{a}R^{c} where R^{a} is an alkyl radical as defined above and R^{c} is a heterocyclyl radical as defined herein. The alkyl radical and the heterocyclyl radical independently may be optionally substituted as defined herein.

As used herein, “aralkyl” refers to a radical of the formula —R^{a}R^{d} where R^{a} is an alkyl group radical as defined herein and R^{d} is an aryl radical as defined herein. The alkyl radical and the aryl radical may be optionally substituted as defined herein.

As used herein, “halo”, “halogen” or “halide” refers to F, Cl, Br or I.

As used herein, “haloalkyl” refers to an alkyl group in which one or more of the hydrogen atoms are replaced by halogen. Such groups include, but are not limited to, chloromethyl, trifluoromethyl and 1-chloro-2-fluoroethyl.

As used herein, “hydrazone” refers to a divalent group such as ═NNR^{t} which is attached to a carbon atom of another group, forming a double bond, wherein R^{t} is hydrogen or alkyl.

As used herein, “imino” refers to a divalent group such as ═NR, which is attached to a carbon atom of another group, forming a double bond, wherein R is hydrogen or alkyl.

Unless stated otherwise, optionally substituted alkyl, alkenyl and alkynyl refer to alkyl, alkenyl or alkynyl radicals, as defined herein, that may be optionally substituted by one or more (e.g., 1-6, 1-4, 1-2, or 1) substituents independently selected from the group consisting of nitro, halo, azido, cyano, cycloalkyl, heteroaryl,

heterocyclyl, —ORₓ, —N(R_{y})(R_{z}), —SRₓ, —C(J)Rₓ, —C(J)ORₓ, —C(J)N(R_{y})(R_{z}), —C(J)SRₓ, —S(O)ₜRₓ (where t is 1 or 2), —OC(J)Rₓ, —OC(J)ORₓ, —OC(J)N(R_{y})(R_{z}), —OC(J)SRₓ, —N(Rₓ)C(J)Rₓ, —N(Rₓ)C(J)ORₓ, —N(Rₓ)C(J)N(R_{y})(R_{z}), —N(Rₓ)C(J)SRₓ, —Si(R_{w})₃, —N(Rₓ)S(O)₂R_{w}, —N(Rₓ)S(O)₂N(R_{y})(R_{z}), —S(O)₂N(R_{y})(R_{z}), —N(Rₓ)C(J)Rₓ, —P(O)(R_{V})₂, —OP(O)(Rᵥ)₂, —C(J)N(Rₓ)S(O)₂Rₓ, —C(J)N(R^{x})N(Rₓ)S(O)₂Rₓ, —C(Rₓ)═N(ORₓ), and —C(Rₓ)═NN(R_{y})(R_{z}), wherein each Rₓ is independently hydrogen, alkyl, alkenyl, alkynyl, cycloalkyl, cycloalkylalkyl, heterocyclyl, heterocyclylalkyl, aryl, aralkyl, heteroaryl, or heteroaralkyl; R_{y} and R_{z} are each independently hydrogen, alkyl, alkenyl, alkynyl, cycloalkyl, cycloalkylalkyl, heterocyclyl, heterocyclylalkyl, aryl, aralkyl, heteroaryl, or heteroaralkyl; or R_{y} and R_{z}, together with the nitrogen atom to which they are attached, form a heterocyclyl or heteroaryl; each R_{w} is independently alkyl, alkenyl, alkynyl, cycloalkyl, cycloalkylalkyl, heterocyclyl, heterocyclylalkyl, aryl, aralkyl, heteroaryl, or heteroaralkyl; each Rᵥ is independently alkyl, alkenyl, alkynyl, cycloalkyl, cycloalkylalkyl, heterocyclyl, heterocyclylalkyl, aryl, aralkyl, heteroaryl, heteroaralkyl, hydroxy, —ORₓ or —N(R_{y})(R_{z}); and each J is independently O, NRₓ or S.

In some embodiments, optionally substituted alkyl, alkenyl and alkynyl refer to alkyl, alkenyl or alkynyl radicals, as defined herein, that may be optionally substituted by one or more (e.g., 1-6, 1-4, 1-2, or 1) substituents independently selected from the group consisting of halo, cyano, cycloalkyl, heteroaryl,

heterocyclyl, —ORₓ, —N(R_{y})(R_{z}), —SRₓ, —C(J)Rₓ, —C(J)ORₓ, —C(J)N(R_{y})(R_{z}), —C(J)SRₓ, —S(O)ₜRₓ (where t is 1 or 2), —OC(J)Rₓ, —OC(J)ORₓ, —OC(J)N(R_{y})(R_{z}), —OC(J)SRₓ, —N(Rₓ)C(J)Rₓ, —N(Rₓ)C(J)ORₓ, —N(Rₓ)C(J)N(R_{y})(R_{z}), —N(Rₓ)C(J)SRₓ, —N(Rₓ)S(O)₂R_{w}, —N(Rₓ)S(O)₂N(R_{y})(R_{z}), —S(O)₂N(R_{y})(R_{z}), —N(Rₓ)C(J)Rₓ, —C(J)N(Rₓ)S(O)₂ Rₓ, and —C(J)N(Rₓ)N(Rₓ)S(O)₂Rₓ, wherein each Rₓ is independently hydrogen, alkyl, alkenyl, alkynyl, cycloalkyl, cycloalkylalkyl, heterocyclyl, heterocyclylalkyl, aryl, aralkyl, heteroaryl, or heteroaralkyl; R_{y} and R_{z} are each independently hydrogen, alkyl, alkenyl, alkynyl, cycloalkyl, cycloalkylalkyl, heterocyclyl, heterocyclylalkyl, aryl, aralkyl, heteroaryl, or heteroaralkyl; or R_{y} and R_{z}, together with the nitrogen atom to which they are attached, form a heterocyclyl or heteroaryl; each R_{w} is independently alkyl, alkenyl, alkynyl, cycloalkyl, cycloalkylalkyl, heterocyclyl, heterocyclylalkyl, aryl, aralkyl, heteroaryl, or heteroaralkyl; each Rᵥ is independently alkyl, alkenyl, alkynyl, cycloalkyl, cycloalkylalkyl, heterocyclyl, heterocyclylalkyl, aryl, aralkyl, heteroaryl, heteroaralkyl, hydroxy, —ORₓ or —N(R_{y})(R_{z}); and each J is independently O, NRₓ or S.

In some embodiments, optionally substituted alkyl, alkenyl and alkynyl refer to alkyl, alkenyl or alkynyl radicals, as defined herein, that may be optionally substituted by one or more (e.g., 1-6, 1-4, 1-2, or 1) substituents independently selected from the group consisting of halo, cyano, cycloalkyl, heteroaryl,

heterocyclyl, —ORₓ, —N(R_{y})(R_{z}), —C(J)Rₓ, —C(J)ORₓ, —C(J)N(R_{y})(R_{z}), —OC(J)Rₓ, —OC(J)ORₓ, —OC(J)N(R_{y})(R_{z}), —N(Rₓ)C(J)Rₓ, —N(Rₓ)C(J)ORₓ, —N(Rₓ)C(J)N(R_{y})(R_{z}), and —N(Rₓ)C(J)Rₓ, wherein each Rₓ is independently hydrogen, alkyl, alkenyl, alkynyl, cycloalkyl, cycloalkylalkyl, heterocyclyl, heterocyclylalkyl, aryl, aralkyl, heteroaryl, or heteroaralkyl; R_{y} and R_{z} are each independently hydrogen, alkyl, alkenyl, alkynyl, cycloalkyl, cycloalkylalkyl, heterocyclyl, heterocyclylalkyl, aryl, aralkyl, heteroaryl, or heteroaralkyl; or R_{y} and R_{z}, together with the nitrogen atom to which they are attached, form a heterocyclyl or heteroaryl; each R_{w} is independently alkyl, alkenyl, alkynyl, cycloalkyl, cycloalkylalkyl, heterocyclyl, heterocyclylalkyl, aryl, aralkyl, heteroaryl, or heteroaralkyl; each Rᵥ is independently alkyl, alkenyl, alkynyl, cycloalkyl, cycloalkylalkyl, heterocyclyl, heterocyclylalkyl, aryl, aralkyl, heteroaryl, heteroaralkyl, hydroxy, —ORₓ or —N(R_{y})(R_{z}); and each J is independently O, NRₓ or S.

In some embodiments, optionally substituted alkyl, alkenyl and alkynyl refer to alkyl, alkenyl or alkynyl radicals, as defined herein, that may be optionally substituted by one or more (e.g., 1-6, 1-4, 1-2, or 1) substituents independently selected from the group consisting of halo, cyano, cycloalkyl, heteroaryl,

heterocyclyl, —ORₓ, —N(R_{y})(R_{z}), —C(J)Rₓ, —C(J)ORₓ, —C(J)N(R_{y})(R_{z}), —OC(J)Rₓ, —OC(J)ORₓ, —OC(J)N(R_{y})(R_{z}), —N(Rₓ)C(J)Rₓ, —N(Rₓ)C(J)ORₓ, —N(Rₓ)C(J)N(R_{y})(R_{z}), and —N(Rₓ)C(J)Rₓ, wherein each Rₓ is independently hydrogen, alkyl, cycloalkyl, cycloalkylalkyl, heterocyclyl, heterocyclylalkyl, aryl, aralkyl, heteroaryl, or heteroaralkyl; R_{y} and R_{z} are each independently hydrogen, alkyl, cycloalkyl, cycloalkylalkyl, heterocyclyl, heterocyclylalkyl, aryl, aralkyl, heteroaryl, or heteroaralkyl; or R_{y} and R_{z}, together with the nitrogen atom to which they are attached, form a heterocyclyl or heteroaryl; each R_{w} is independently alkyl, cycloalkyl, cycloalkylalkyl, heterocyclyl, heterocyclylalkyl, aryl, aralkyl, heteroaryl, or heteroaralkyl; each Rᵥ is independently alkyl, cycloalkyl, cycloalkylalkyl, heterocyclyl, heterocyclylalkyl, aryl, aralkyl, heteroaryl, heteroaralkyl, hydroxy, —ORₓ or —N(R_{y})(R_{z}); and each J is independently O, NRₓ or S.

In some embodiments, optionally substituted alkyl, alkenyl and alkynyl refer to alkyl, alkenyl or alkynyl radicals, as defined herein, that may be optionally substituted by one or more (e.g., 1-6, 1-4, 1-2, or 1) substituents independently selected from the group consisting of halo, cyano, cycloalkyl, heteroaryl, heterocyclyl, —C(J)Rₓ, —C(J)ORₓ, and —C(J)N(R_{y})(R_{z}), wherein each Rₓ is independently hydrogen, alkyl, cycloalkyl, cycloalkylalkyl, heterocyclyl, heterocyclylalkyl, aryl, aralkyl, heteroaryl, or heteroaralkyl; R_{y} and R_{z} are each independently hydrogen, alkyl, cycloalkyl, cycloalkylalkyl, heterocyclyl, heterocyclylalkyl, aryl, aralkyl, heteroaryl, or heteroaralkyl; or R_{y} and R_{z}, together with the nitrogen atom to which they are attached, form a heterocyclyl or heteroaryl; each R_{w} is independently alkyl, cycloalkyl, cycloalkylalkyl, heterocyclyl, heterocyclylalkyl, aryl, aralkyl, heteroaryl, or heteroaralkyl; each Rᵥ is independently alkyl, cycloalkyl, cycloalkylalkyl, heterocyclyl, heterocyclylalkyl, aryl, aralkyl, heteroaryl, heteroaralkyl, hydroxy, —ORₓ or —N(R_{y})(R_{z}); and each J is independently O, NRₓ or S.

Unless stated otherwise, “optionally substituted aryl”, “optionally substituted cycloalkyl”, “optionally substituted heteroaryl”, and “optionally substituted heterocyclyl” refer to aryl, cycloalkyl, heterocyclyl, and heteroaryl radicals, respectively, as defined herein, that are optionally substituted by one or more (e.g., 1-6, 1-4, 1-2, or 1) substituents selected from the group consisting of nitro, halo, azido, cyano, alkyl, haloalkyl, alkenyl, alkynyl, cycloalkyl, cycloalkylalkyl, heterocyclyl, heterocyclylalkyl, aryl, aralkyl, heteroaryl, heteroaralkyl, —Rᵤ—ORₓ, —Rᵤ—N(R_{y})(R_{z}), —Rᵤ—SRₓ, —Rᵤ—C(J)Rₓ, —Rᵤ—C(J)ORₓ, —Rᵤ—C(J)N(R_{y})(R_{z}), —Rᵤ—C(J)SRₓ, —Rᵤ—S(O)ₜRₓ (where t is 1 or 2), —Rᵤ—OC(J)Rₓ, —Rᵤ—OC(J)ORₓ, —Rᵤ—OC(J)N(R_{y})(R_{z}), —Rᵤ—OC(J)SRₓ, —Rᵤ—N(Rₓ)C(J)Rₓ, —Rᵤ—N(Rₓ)C(J)ORₓ, —Rᵤ—N(Rₓ)C(J)N(R_{y})(R_{z}), —Rᵤ—N(Rₓ)C(J)SRₓ, —Rᵤ —Si(R_{w})₃, —Rᵤ—N(Rₓ)S(O)₂R_{w}, —Rᵤ—N(Rₓ)S(O)₂N(R_{y})(R_{z}), —Rᵤ—S(O)₂N(R_{y})(R_{z}), —Rᵤ —N(Rₓ)C(J)Rₓ, —Rᵤ—P(O)(Rᵥ)₂, —Rᵤ—OP(O)(Rᵥ)₂, —Rᵤ—C(J)N(Rₓ)S(O)₂Rₓ, —Rᵤ —C(J)N(Rₓ)N(Rₓ)S(O)₂Rₓ, —Rᵤ—C(Rₓ)=N(ORₓ), and —Rᵤ—C(Rₓ)=NN(R_{y})(R_{z}), wherein each Rᵤ is independently alkylene or a direct bond; each Rᵥ is independently alkyl, alkenyl, alkynyl, cycloalkyl, cycloalkylalkyl, heterocyclyl, heterocyclylalkyl, aryl, aralkyl, heteroaryl, heteroaralkyl, hydroxy, —ORₓ or —N(R_{y})(R_{z}); each R_{w} is independently alkyl, alkenyl, alkynyl, cycloalkyl, cycloalkylalkyl, heterocyclyl, heterocyclylalkyl, aryl, aralkyl, heteroaryl, or heteroaralkyl; each Rₓ is independently hydrogen, alkyl, alkenyl, alkynyl, cycloalkyl, cycloalkylalkyl, heterocyclyl, heterocyclylalkyl, aryl, aralkyl, heteroaryl, or heteroaralkyl; R_{y} and R_{z} are each independently hydrogen, alkyl, alkenyl, alkynyl, cycloalkyl, cycloalkylalkyl, heterocyclyl, heterocyclylalkyl, aryl, aralkyl, heteroaryl, or heteroaralkyl; or R_{y} and R_{z}, together with the nitrogen atom to which they are attached, form a heterocyclyl or heteroaryl; and each J is O, NRₓ or S.

In some embodiments, “optionally substituted aryl”, “optionally substituted cycloalkyl”, “optionally substituted heteroaryl”, and “optionally substituted heterocyclyl” refer to aryl, cycloalkyl, heterocyclyl, and heteroaryl radicals, respectively, as defined herein, that are optionally substituted by one or more (e.g., 1-6, 1-4, 1-2, or 1) substituents selected from the group consisting of nitro, halo, cyano, alkyl, haloalkyl, alkenyl, alkynyl, cycloalkyl, cycloalkylalkyl, heterocyclyl, heterocyclylalkyl, aryl, aralkyl, heteroaryl, heteroaralkyl, —Rᵤ—ORₓ, —Rᵤ—N(R_{y})(R_{z}), —Rᵤ—SRₓ, —Rᵤ—C(J)Rₓ, —Rᵤ—C(J)ORₓ, —Rᵤ—C(J)N(R_{y})(R_{z}), —Rᵤ—C(J)SRₓ, —Rᵤ—S(O)ₜRₓ, (where t is 1 or 2), —Rᵤ—OC(J)Rₓ, —Rᵤ—OC(J)ORₓ, —Rᵤ—OC(J)N(R_{y})(R_{z}), —Rᵤ—OC(J)SRₓ, —Rᵤ—N(Rₓ)C(J)Rₓ, —Rᵤ—N(Rₓ)C(J)ORₓ, —Rᵤ—N(Rₓ)C(J)N(R_{y})(R^{z}), —Rᵤ—N(Rₓ)C(J)SRₓ, —Rᵤ—N(Rₓ)S(O)₂R_{w}, —Rᵤ—N(Rₓ)S(O)₂N(R_{y})(R_{z}), —Rᵤ—S(O)₂N(R_{y})(R_{z}), —Rᵤ —N(Rₓ)C(J)Rₓ, —Rᵤ—C(J)N(Rₓ)S(O)₂Rₓ, and —Rᵤ —C(J)N(Rₓ)N(Rₓ)S(O)₂Rₓ, wherein each Rᵤ is independently alkylene or a direct bond; each Rᵥ is independently alkyl, alkenyl, alkynyl, cycloalkyl, cycloalkylalkyl, heterocyclyl, heterocyclylalkyl, aryl, aralkyl, heteroaryl, heteroaralkyl, hydroxy, —ORₓ or —N(R_{y})(R_{z}); each R_{w} is independently alkyl, alkenyl, alkynyl, cycloalkyl, cycloalkylalkyl, heterocyclyl, heterocyclylalkyl, aryl, aralkyl, heteroaryl, or heteroaralkyl; each Rₓ is independently hydrogen, alkyl, alkenyl, alkynyl, cycloalkyl, cycloalkylalkyl, heterocyclyl, heterocyclylalkyl, aryl, aralkyl, heteroaryl, or heteroaralkyl; R_{y} and R_{z} are each independently hydrogen, alkyl, alkenyl, alkynyl, cycloalkyl, cycloalkylalkyl, heterocyclyl, heterocyclylalkyl, aryl, aralkyl, heteroaryl, or heteroaralkyl; or R_{y} and R_{z}, together with the nitrogen atom to which they are attached, form a heterocyclyl or heteroaryl; and each J is O, NRₓ or S.

In some embodiments, “optionally substituted aryl”, “optionally substituted cycloalkyl”, “optionally substituted heteroaryl”, and “optionally substituted heterocyclyl” refer to aryl, cycloalkyl, heterocyclyl, and heteroaryl radicals, respectively, as defined herein, that are optionally substituted by one or more (e.g., 1-6, 1-4, 1-2, or 1) substituents selected from the group consisting of nitro, halo, cyano, alkyl, haloalkyl, alkenyl, alkynyl, cycloalkyl, cycloalkylalkyl, heterocyclyl, heterocyclylalkyl, aryl, aralkyl, heteroaryl, heteroaralkyl, —Rᵤ—ORₓ, —Rᵤ—N(R_{y})(R_{z}), —Rᵤ—C(J)Rₓ, —Rᵤ—C(J)ORₓ, —Rᵤ—C(J)N(R_{y})(R^{z}), —Rᵤ—OC(J)Rₓ, —Rᵤ—OC(J)ORₓ, —Rᵤ—OC(J)N(R_{y})(R_{z}), —Rᵤ— N(Rₓ)C(J)Rₓ, —Rᵤ—N(Rₓ)C(J)ORₓ, —Rᵤ—N(Rₓ)C(J)N(R_{y})(R_{z}), and —Rᵤ —N(Rₓ)C(J)Rₓ, wherein each Rᵤ is independently alkylene or a direct bond; each Rᵥ is independently alkyl, alkenyl, alkynyl, cycloalkyl, cycloalkylalkyl, heterocyclyl, heterocyclylalkyl, aryl, aralkyl, heteroaryl, heteroaralkyl, hydroxy, —ORₓ or —N(R_{y})(R_{z}); each R_{w} is independently alkyl, alkenyl, alkynyl, cycloalkyl, cycloalkylalkyl, heterocyclyl, heterocyclylalkyl, aryl, aralkyl, heteroaryl, or heteroaralkyl; each Rₓ is independently hydrogen, alkyl, alkenyl, alkynyl, cycloalkyl, cycloalkylalkyl, heterocyclyl, heterocyclylalkyl, aryl, aralkyl, heteroaryl, or heteroaralkyl; R_{y} and R_{z} are each independently hydrogen, alkyl, alkenyl, alkynyl, cycloalkyl, cycloalkylalkyl, heterocyclyl, heterocyclylalkyl, aryl, aralkyl, heteroaryl, or heteroaralkyl; or R_{y} and R_{z}, together with the nitrogen atom to which they are attached, form a heterocyclyl or heteroaryl; and each J is O, NRₓ or S.

In some embodiments, “optionally substituted aryl”, “optionally substituted cycloalkyl”, “optionally substituted heteroaryl”, and “optionally substituted heterocyclyl” refer to aryl, cycloalkyl, heterocyclyl, and heteroaryl radicals, respectively, as defined herein, that are optionally substituted by one or more (e.g., 1-6, 1-4, 1-2, or 1) substituents selected from the group consisting of nitro, halo, cyano, alkyl, haloalkyl, cycloalkyl, cycloalkylalkyl, heterocyclyl, heterocyclylalkyl, aryl, aralkyl, heteroaryl, heteroaralkyl, —Rᵤ—ORₓ, —Rᵤ—N(R_{y})(R_{z}), —Rᵤ—C(J)Rₓ, —Rᵤ—C(J)ORₓ, —Rᵤ—C(J)N(R_{y})(R_{z}), —Rᵤ—OC(J)Rₓ, —Rᵤ—OC(J)ORₓ, —Rᵤ—OC(J)N(R_{y})(R_{z}), —Rᵤ—N(Rₓ)C(J)Rₓ, —Rᵤ—N(Rₓ)C(J)ORₓ, —Rᵤ—N(Rₓ)C(J)N(R_{y})(R_{z}), and —Rᵤ —N(Rₓ)C(J)Rₓ, wherein each Rᵤ is independently alkylene or a direct bond; each Rᵥ is independently alkyl, alkenyl, alkynyl, cycloalkyl, cycloalkylalkyl, heterocyclyl, heterocyclylalkyl, aryl, aralkyl, heteroaryl, heteroaralkyl, hydroxy, —ORₓ or —N(R_{y})(R_{z}); each R_{w} is independently alkyl, cycloalkyl, cycloalkylalkyl, heterocyclyl, heterocyclylalkyl, aryl, aralkyl, heteroaryl, or heteroaralkyl; each Rₓ is independently hydrogen, alkyl, cycloalkyl, cycloalkylalkyl, heterocyclyl, heterocyclylalkyl, aryl, aralkyl, heteroaryl, or heteroaralkyl; R_{y} and R_{z} are each independently hydrogen, alkyl, cycloalkyl, cycloalkylalkyl, heterocyclyl, heterocyclylalkyl, aryl, aralkyl, heteroaryl, or heteroaralkyl; or R_{y} and R_{z}, together with the nitrogen atom to which they are attached, form a heterocyclyl or heteroaryl; and each J is O, NRₓ or S.

Unless stated otherwise specifically in the specification, it is understood that the substitution can occur on any atom of the aryl, aralkyl, cycloalkyl, heterocyclyl, and heteroaryl groups. It will be understood by those skilled in the art, with respect to any group containing one or more substituents, that such groups are not intended to introduce any substitution or substitution patterns that are sterically impractical, synthetically non-feasible and/or inherently unstable.

Optionally substituted cycloalkyl and optionally substituted heterocyclyl may additionally be substituted with oxo, thioxo, imino, oxime or hydrazone, on a saturated carbon of their respective ring system.

As used herein, “oxime” refers to a divalent group such as ═N—OH, which is attached to a carbon atom of another group, forming a double bond.

As used herein, “oxo” refers to an oxygen atom doubly bonded to a carbon.

As used herein, “thioxo” refers to a sulfur atom doubly bonded to a carbon.

Where the number of any given substituent is not specified (e.g., haloalkyl), there may be one or more substituents present. For example, “haloalkyl” may include one or more of the same or different halogens.

As used herein, the abbreviations for any protective groups, amino acids and other compounds, are, unless indicated otherwise, in accord with their common usage, recognized abbreviations or the IUPAC-IUB Commission on Biochemical Nomenclature (see, (1972) Biochem. 11:942-944).

If employed herein, the following terms have their accepted meaning in the chemical literature.
BOP benzotriazol-1-yloxy-tris(dimethylamino)-phosphonium hexafluorophosphate
DMAP 4-(dimethylamino) pyridine
DMF N,N-dimethylformamide
DMSO dimethylsulfoxide
EDC 1-(3-dimethylaminopropyl)-3-ethylcarbodiimide hydrochloride
HATU O-(7-azabenzotriazol-1-yl)-N,N,N′,N′-tetramethyluronium hexafluorophosphate
HoAc acetic acid
HOBt 1-hydroxybenzotriazole hydrate
NMP 1-methyl-2-pyrrolidinone
THF tetrahydrofuran
TFA trifluoroacetic acid

Provided is at least one chemical entity chosen from compounds of Formula I

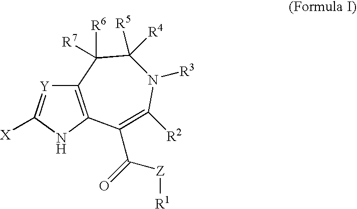

and pharmaceutically acceptable salts thereof, wherein
X is chosen from CN, CF₃, CF₂H, S(O)ₙR⁸, and S(O)₂N(R⁹)R¹⁰;
n is 0, 1, or 2;
Y is chosen from CR¹¹ and N;
Z is chosen from O and NH;
R¹ is chosen from optionally substituted alkyl, optionally substituted cycloalkyl, optionally substituted heterocyclyl, optionally substituted aryl, and optionally substituted heteroaryl;
R² is chosen from hydrogen and optionally substituted alkyl;
R³ is chosen from —C(O)R¹² and —C(O)N(R⁹)R¹⁰;
R⁴, R⁵, R⁶ and R⁷ are independently chosen from hydrogen and optionally substituted alkyl, or any two of R⁴, R⁵, R⁶ and R⁷, together with the atoms to which they are attached, form an optionally substituted cycloalkyl or optionally substituted heterocyclyl ring;
R⁸ is chosen from optionally substituted alkyl, optionally substituted cycloalkyl, optionally substituted heterocyclyl, optionally substituted aryl, and optionally substituted heteroaryl;
each occurrence of R⁹ and R¹⁰ is independently chosen from hydrogen, optionally substituted aryl, optionally substituted heteroaryl, optionally substituted alkyl, optionally substituted cycloalkyl, and optionally substituted heterocyclyl, or R⁹ and R¹⁰, together with the atoms to which they are attached, form an optionally substituted heterocyclyl ring;
R¹¹ is chosen from hydrogen and lower alkyl; and
R¹² is chosen from hydrogen, optionally substituted aryl, optionally substituted heteroaryl, optionally substituted alkyl, optionally substituted cycloalkyl, and optionally substituted heterocyclyl.

In some embodiments, R⁴ and R⁵ are independently chosen from hydrogen and optionally substituted alkyl. In some embodiments, R⁴ and R⁵ are independently chosen from hydrogen and optionally substituted lower alkyl. In some embodiments, R⁴ and R⁵ are independently chosen from hydrogen and lower alkyl. In some embodiments, one of R⁴ and R⁵ is hydrogen and the other is lower alkyl. In some embodiments, R⁴ and R⁵ are hydrogen.

In some embodiments, Y is N. In some embodiments, Y is CR¹¹. In some embodiments, R¹¹ is chosen from hydrogen and methyl. In some embodiments, R¹¹ is hydrogen.

In some embodiments, X is chosen from CN, CF₃, and CF₂H. In some embodiments, X is CN.

In some embodiments, R⁶ and R⁷ are independently chosen from hydrogen and optionally substituted alkyl. In some embodiments, R⁶ and R⁷ are independently chosen from hydrogen and optionally substituted lower alkyl. In some embodiments, R⁶ and R⁷ are independently chosen from hydrogen and lower alkyl. In some embodiments, one of R⁶ and R⁷ is hydrogen and the other is lower alkyl. In some embodiments, one of R⁶ and R⁷ is hydrogen and the other is methyl. In some embodiments, R⁶ and R⁷ are independently lower alkyl. In some embodiments, R⁶ and R⁷ are methyl.

In some embodiments, R¹ is optionally substituted alkyl. In some embodiments, R¹ is optionally substituted lower alkyl. In some embodiments, R¹ is lower alkyl. In some embodiments, R¹ is propyl. In some embodiments, R¹ is iso-propyl.

In some embodiments, R² is chosen from hydrogen and optionally substituted lower alkyl. In some embodiments, R² is chosen from hydrogen and lower alkyl. In some embodiments, R² is hydrogen.

In some embodiments, R³ is —C(O)R¹². In some embodiments, R¹² is chosen from optionally substituted aryl, optionally substituted heteroaryl, optionally substituted cycloalkyl, and optionally substituted heterocyclyl. In some embodiments, R¹² is chosen from cycloalkyl, heterocyclyl, phenyl, and heteroaryl, each of which is optionally substituted with one, two or three groups independently chosen from halo, cyano, lower alkyl, lower alkyl substituted with one, two, or three halo groups, hydroxy, and lower alkoxy. In some embodiments, R¹² is chosen from cyclohexyl, phenyl, and thienyl, each of which is optionally substituted with one, two or three groups independently chosen from halo, cyano, lower alkyl, lower alkyl substituted with one, two, or three halo groups, hydroxy, and lower alkoxy. In some embodiments, R¹² is chosen from hydrogen, 3,4-difluorophenyl, cyclohexyl, 3-fluorophenyl, 4-fluorophenyl, 4-cyanophenyl, 3-chlorophenyl, thienyl, and 3-trifluorophenyl.

In some embodiments, R³ is —C(O)N(R⁹)R¹⁰. In some embodiments, each occurrence of R⁹ and R¹⁰ is independently chosen from hydrogen and optionally substituted alkyl. In some embodiments, each occurrence of R⁹ and R¹⁰ is independently chosen from hydrogen and alkyl. In some embodiments, each occurrence of R⁹ and R¹⁰ is independently chosen from hydrogen and lower alkyl.

In some embodiments, Z is O. In some embodiments, Z is NH.

Also provided is at least one chemical entity chosen from compounds of Formula II

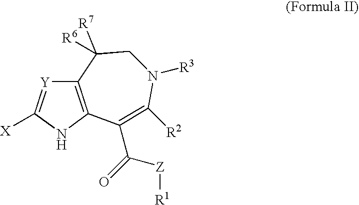

and pharmaceutically acceptable salts thereof wherein R¹, R², R³, R⁶, R⁷, X, Y, and Z are as described for compounds of Formula I.

Also provided is at least one chemical entity chosen from compounds of Formula III

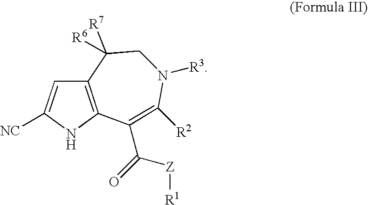

and pharmaceutically acceptable salts thereof wherein R¹, R², R³, R⁶, R⁷, and Z are as described for compounds of Formula I.

In some embodiments, the compound of Formula I is chosen from
Isopropyl 2-cyano-4,4-dimethyl-1,4,5,6-tetrahydropyrrolo[2,3-d]azepine-8-carboxylate;
Isopropyl 2-cyano-6-(3,4-difluorobenzoyl)-4,4-dimethyl-1,4,5,6-tetrahydropyrrolo[2,3-d]azepine-8-carboxylate;
Isopropyl 2-cyano-6-(cyclohexylcarbonyl)-4,4-dimethyl-1,4,5,6-tetrahydropyrrolo[2,3-d]azepine-8-carboxylate;
Isopropyl 2-cyano-6-(3-fluorobenzoyl)-4,4-dimethyl-1,4,5,6-tetrahydropyrrolo[2,3-d]azepine-8-carboxylate;
Isopropyl 2-cyano-6-(4-fluorobenzoyl)-4,4-dimethyl-1,4,5,6-tetrahydropyrrolo[2,3-d]azepine-8-carboxylate;
Isopropyl 2-cyano-6-(4-cyanobenzoyl)-4,4-dimethyl-1,4,5,6-tetrahydropyrrolo[2,3-d]azepine-8-carboxylate;
Isopropyl 6-(3-chlorobenzoyl)-2-cyano-4,4-dimethyl-1,4,5,6-tetrahydropyrrolo[2,3-d]azepine-8-carboxylate;
Isopropyl 2-cyano-4,4-dimethyl-6-(2-thienylcarbonyl)-1,4,5,6-tetrahydropyrrolo[2,3-d]azepine-8-carboxylate;
Isopropyl 2-cyano-4,4-dimethyl-6-[3-(trifluoromethyl)benzoyl]-1,4,5,6-tetrahydropyrrolo[2,3-d]azepine-8-carboxylate;
Isopropyl 2-cyano-4,4-dimethyl-6-(tetrahydro-2H-pyran-4-ylcarbonyl)-1,4,5,6-tetrahydropyrrolo[2,3-d]azepine-8-carboxylate; and
Isopropyl 2′-cyano-6′-(3,4-difluorobenzoyl)-2,3,5,5′,6,6′-hexahydro-1′H-spiro[pyran-4,4′-pyrrolo[2,3-d]azepine]-8′carboxylate.

The pharmaceutical compositions provided herein contain therapeutically effective amounts of one or more of the chemical entities provided herein that may be useful in the prevention or treatment of one or more of the symptoms of diseases or disorders associated with nuclear receptor activity, including the farnesoid X receptor and/or orphan nuclear receptor activity.

Accordingly, also provided are methods of treating at least one disease or disorder in a patient by administering to the patient a therapeutically effective amount of at least one chemical entity disclosed herein. In embodiments the disease or disorder is selected from hyperlipidemia, hypercholesterolemia, hyperlipoproteinemia, hypertriglyceridemia, dyslipidemia, lipodystrophy, atherosclerosis, atherosclerotic disease, atherosclerotic disease events, atherosclerotic cardiovascular disease, Syndrome X, diabetes mellitus, type II diabetes, insulin insensitivity, hyperglycemia, cholestasis and obesity, gallstone disease, acne vulgaris, acneiform skin conditions, Parkinson's disease, cancer, Alzheimer's disease, inflammation, immunological disorders, lipid disorders, obesity, conditions characterized by a perturbed epidermal barrier function, cholestasis, peripheral occlusive disease, ischemic stroke, conditions of disturbed differentiation or excess proliferation of the epidermis or mucous membrane, cardiovascular disorders, diabetic nephropathy, metabolic acidosis, hypertension, myocardial infarction, hypertension and heart failure. In some embodiments the chemical entity is a FXR agonist.

In some embodiments the method further comprises co-administering at least one additional active agent selected from antihyperlipidemic agents, plasma HDL-raising agents, antihypercholesterolemic agents, cholesterol biosynthesis inhibitors, HMG CoA reductase inhibitors, acylcoenzyme A cholesterol acytransferase (ACAT) inhibitors, probucol, raloxifene, nicotinic acid, niacinamide, cholesterol absorption inhibitors, bile acid sequestrants, low density lipoprotein receptor inducers, clofibrate, fenofibrate, benzofibrate, cipofibrate, gemfibrizol, vitamin B₆, vitamin B₁₂, vitamin C, vitamin E, β-blockers, anti-diabetes agents, sulfonylureas, biguanides, thiazolidinediones, activators of PPARα, PPARβ and PPARγ, dehydroepiandrosterone, antiglucocorticoids, TNFα inhibitors, α-glucosidase inhibitors, pramlintide, insulin, angiotensin II antagonists, angiotensin converting enzyme inhibitors, platelet aggregation inhibitors, fibrinogen receptor antagonists, LXR α agonists, partial agonists or antagonists, LXR β agonists, partial agonists or antagonists, phenylpropanolamine, phentermine, diethylpropion, mazindol, fenfiuramine, dexfenfiuramine, phentiramine, β₃ adrenoceptor agonist agents, sibutramine, gastrointestinal lipase inhibitors, neuropeptide γ, enterostatin, cholecytokinin, bombesin, amylin, histamine H3 receptor agonists or antagonists, dopamine D2 receptor agonists or antagonists, melanocyte stimulating hormone, corticotrophin releasing factor, leptin, galanin or gamma amino butyric acid (GABA), aspirin and fibric acid derivatives.

Also provided are methods of treating at least one malignancy in a patient by administering to the patient a therapeutically effective amount of at least one farnesoid X receptor (FXR) agonist disclosed herein. In some embodiments the at least one FXR agonist induces expression of the reversion-inducing-cysteine rich-protein with Kazal motifs (RECK) gene in the patient. In some embodiments the at least one malignancy is selected from hepatocellular carcinoma, colorectal cancer, and breast cancer. In some embodiments the at least one malignancy is characterized by elevated expression of the human epidermal growth factor receptor 2 (HER2/neu) gene. In some embodiments the at least one malignancy is selected from hepatocellular carcinoma, colorectal cancer, breast cancer, gastric cancer, renal cancer, salivary gland cancer, ovarian cancer, uterine body cancer, bladder cancer, and lung cancer. In some embodiments the FXR agonist reduces at least one feature of the malignancy, wherein the at least one feature of the malignancy is selected from invasive activity, metastatic activity, and angiogenic activity of the malignancy. In some embodiments, the method further comprises coadministering at least one of an agent selected from abarelix, aldeleukin, allopurinol, altretamine, amifostine, anastozole, bevacizumab, capecitabine, carboplatin, cisplatin, docetaxel, doxorubicin, erlotinib, exemestane, 5-fluorouracil, fulvestrant, gemcitabine, goserelin acetate, irinotecan, lapatinib ditosylate, letozole, leucovorin, levamisole, oxaliplatin, paclitaxel, panitumumab, pemetrexed disodium, profimer sodium, tamoxifen, topotecan, and trastuzumab. In some embodiments of the method, the FXR agonist does not induce expression of the small heterodimer partner (SHP) gene in the patient.

Also provided are methods of treating at least one disease state characterized by elevated expression of the Lectin-like Oxidized Low-density Lipoprotein Receptor 1 (LOX-1) in a patient by administering to the patient a therapeutically effective amount of at least one farnesoid X receptor (FXR) agonist, where the at least one FXR agonist reduces expression of LOX-1 in the patient. In some embodiments the disease state is further characterized by at least one of endothelial dysfunction and vascular inflammation. In some embodiments the at least one disease state is selected from heart failure, myocardial injury, atherosclerosis, diabetic nephropathy, hypertension, sepsis, osteoarthritis and rheumatoid arthritis. In some embodiments the heart failure comprises at least one of left sided heart failure, right sided heart failure, systolic heart failure, and diastolic heart failure. In some embodiments the myocardial injury comprises at least one of unstable angina and myocardial infarction. In some embodiments the FXR agonist reduces at least one of NF-κB pathway signaling, MAPK pathway signaling, and production of reactive oxygen species in the patient. In some embodiments the FXR agonist increases nitric oxide production in the patient. In some embodiments LOX-1 expression is reduced in at least one tissue of the patient selected from heart, liver, and kidney. In some embodiments LOX-1 expression is reduced in at least one cell type of the patient selected from endothelial cells, macrophages, smooth muscle cells, dendritic cells, cardiac myocytes, and platelets. In some embodiments the level of serum soluble LOX-1 protein in the patient is reduced. In some embodiments expression of at least one LOX-1 target selected from MCP-1, VCAM-1 and ICAM-1 is reduced in the patient. In some embodiments expression of at least one FXR target selected from DDAH1, ASS1, and GTPCH is increased in the patient. In some embodiments the level of assymetric dimethylarginine (ADMA) is reduced in the patient. In some embodiments expression of nitric oxide synthase is increased in the patient. In some embodiments the LOX-1 expression level in the patient is reduced to about the level of LOX-1 expression in the absence of the disease state. In some embodiments the LOX-1 expression level in the patient is reduced to below about a threshold level of LOX-1 expression. In some embodiments the threshold level of LOX-1 expression is higher than the level of LOX-1 expression in the absence of the disease state.

Also provided are methods of treating nonalcoholic fatty liver disease in a patient, by administering to the patient a therapeutically effective amount of at least one farnesoid X receptor (FXR) agonist disclosed herein. In some embodiments the FXR agonist modulates at least one feature of nonalcoholic fatty liver disease selected from neutral lipid deposition, intracellular lipid droplet formation, inflammatory cell infiltration, inflammatory cholangitis, portal inflammation, liver enzyme activity, liver enzyme expression, inflammatory mediator level, and inflammatory gene expression. In some embodiments the FXR agonist reduces at least one of the following features: neutral lipid deposition, intracellular lipid droplet formation, inflammatory cell infiltration, inflammatory cholangitis, and portal inflammation. In some embodiments the liver enzyme activity is alanine aminotransferase (ALT) activity and the FXR agonist reduces ALT activity. In some embodiments the liver enzyme expression is fatty acid synthase (FAS) expression and the FXR agonist induces FAS expression. In some embodiments the inflammatory mediator is monocyte chemotactic protein-1 (MCP-1) and wherein the FXR agonist reduces the level of MCP-1. In some embodiments the FXR agonist reduces the expression of at least one inflammatory gene and wherein the inflammatory gene is selected from one or more of the following: vascular cell adhesion molecule 1 (VCAM-1), intercellular adhesion molecule-1 (ICAM-1), and tumor necrosis factor α (TN Fα).

Further, the pharmaceutical compositions provided herein contain therapeutically effective amounts of one or more of the chemical entities provided herein that may be useful in the prevention or treatment of one or more of the symptoms of diseases or disorders that are not directly associated with a nuclear receptor, but for which a complication of the disease or disorder is treatable with claimed compounds and compositions. By way of example, without limitation, Cystic Fibrosis is not typically associated with a nuclear receptor activity, but can result in cholestasis, which may be treated with the subject compounds and compositions.

The compositions contain one or more compounds provided herein. The compounds are formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. Typically the compounds described above are formulated into pharmaceutical compositions using techniques and procedures well known in the art (see, e.g., Ansel Introduction to Pharmaceutical Dosage Forms, Fourth Edition 1985, 126).

In the compositions, effective concentrations of at least one chemical entity described herein is mixed with a suitable pharmaceutical carrier or vehicle. The compounds may be derivatized as the corresponding salts, esters, enol ethers or esters, acids, bases, solvates, hydrates or prodrugs prior to formulation, as described above. The concentrations of the compounds in the compositions are effective for delivery of an amount, upon administration, that treats or prevents one or more of the symptoms of diseases or disorders associated with nuclear receptor activity or in which nuclear receptor activity is implicated.

Typically, the compositions are formulated for single dosage administration. To formulate a composition, the weight fraction of compound is dissolved, suspended, dispersed or otherwise mixed in a selected vehicle at an effective concentration such that the treated condition is relieved or ameliorated. Pharmaceutical carriers or vehicles suitable for administration of the compounds provided herein include any such carriers known to those skilled in the art to be suitable for the particular mode of administration.

In addition, the chemical entities described herein may be formulated as the sole pharmaceutically active ingredient in the composition or may be combined with other active ingredients. Liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

The active compound is included in the pharmaceutically acceptable carrier in an amount sufficient to exert a therapeutically useful effect in the absence of undesirable side effects on the patient treated. The therapeutically effective concentration may be determined empirically by testing the compounds in in vitro and in vivo systems described herein and in International Patent Application Publication Nos. 99/27365 and 00/25134 and then extrapolated therefrom for dosages for humans.

The concentration of active compound in the pharmaceutical composition will depend on absorption, inactivation and excretion rates of the active compound, the physicochemical characteristics of the compound, the dosage schedule, and amount administered as well as other factors known to those of skill in the art. For example, the amount that is delivered is sufficient to ameliorate one or more of the symptoms of diseases or disorders associated with nuclear receptor activity or in which nuclear receptor activity is implicated, as described herein.

Typically a therapeutically effective dosage should produce a serum concentration of the chemical entities described herein of from about 0.1 ng/ml to about 50-100 μg/ml. The pharmaceutical compositions typically should provide a dosage of from about 0.001 mg to about 2000 mg of compound per kilogram of body weight per day. Pharmaceutical dosage unit forms are prepared to provide from about 1 mg to about 1000 mg, such as from about 10 to about 500 mg of the chemical entities described herein per dosage unit form.

The chemical entities described herein may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Thus, effective concentrations or amounts of at least one chemical entity described herein is mixed with a suitable pharmaceutical carrier or vehicle for systemic, topical or local administration to form pharmaceutical compositions. The at least one chemical entity is included in an amount effective for treating or preventing diseases or disorders associated with nuclear receptor activity or in which nuclear receptor activity is implicated, as described herein. The concentration of the chemical entity in the composition will depend on absorption, inactivation, excretion rates of the active compound, the dosage schedule, amount administered, particular formulation as well as other factors known to those of skill in the art.

The compositions are intended to be administered by a suitable route, including orally, parenterally, rectally, topically and locally. For oral administration, capsules and tablets may be used. The compositions are in liquid, semi-liquid or solid form and are formulated in a manner suitable for each route of administration. Modes of administration include parenteral and oral modes of administration.

Solutions or suspensions used for parenteral, intradermal, subcutaneous, or topical application can include any of the following components: a sterile diluent, such as water for injection, saline solution, fixed oil, polyethylene glycol, glycerine, propylene glycol or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampoules, disposable syringes or single or multiple dose vials made of glass, plastic or other suitable material.

In instances in which the compounds exhibit insufficient solubility, methods for solubilizing compounds may be used. Such methods are known to those of skill in this art, and include, but are not limited to, using co-solvents, such as dimethylsulfoxide (DMSO), using surfactants, such as TWEEN®, or dissolution in aqueous sodium bicarbonate.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the chemical entity. The chemical entities described herein are typically formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

The composition can contain along with the chemical entities described herein: a diluent such as lactose, sucrose, dicalcium phosphate, or carboxymethylcellulose; a lubricant, such as magnesium stearate, calcium stearate and talc; and a binder such as starch, natural gums, such as gum acacia gelatin, glucose, molasses, polyvinylpyrrolidone, celluloses and derivatives thereof, povidone, crospovidones and other such binders known to those of skill in the art. Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, or solubilizing agents, pH buffering agents and the like, for example, acetate, sodium citrate, cyclodextrin derivatives, sorbitan monolaurate, triethanolamine sodium acetate, triethanolamine oleate, and other such agents. Actual methods of preparing such dosage forms are known, or will be apparent, to those skilled in this art; for example, see Remington's Pharmaceutical Sciences, Mack Publishing Company, Easton, Pa., 15th Edition, 1975. The composition or formulation to be administered will, in any event, contain a quantity of the active compound in an amount sufficient to alleviate the symptoms of the treated subject.

Dosage forms or compositions containing the chemical entities described herein in the range of 0.005% to 100% with the balance made up from non-toxic carrier may be prepared. For oral administration, a pharmaceutically acceptable non-toxic composition is formed by the incorporation of any of the normally employed excipients, such as, for example pharmaceutical grades of mannitol, lactose, starch, magnesium stearate, talcum, cellulose derivatives, sodium crosscarmellose, glucose, sucrose, magnesium carbonate or sodium saccharin. Such compositions include solutions, suspensions, tablets, capsules, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of these compositions are known to those skilled in the art. The contemplated compositions may contain 0.001%-100% of chemical entities described herein, such as 0.1-85%, for example, 75-95%.

The chemical entities described herein may be prepared with carriers that protect the compound against rapid elimination from the body, such as time release formulations or coatings. The compositions may include other active compounds to obtain desired combinations of properties. The chemical entities described herein may also be advantageously administered for therapeutic or prophylactic purposes together with another pharmacological agent known in the general art to be of value in treating one or more of the diseases or medical conditions referred to hereinabove, such as diseases or disorders associated with nuclear receptor activity or in which nuclear receptor activity is implicated. It is to be understood that such combination therapy constitutes a further aspect of the compositions and methods of treatment provided herein.

Oral pharmaceutical dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

In certain embodiments, the formulations are solid dosage forms, such ascapsules or tablets. The tablets, pills, capsules, troches and the like can contain any of the following ingredients, or compounds of a similar nature: a binder; a diluent; a disintegrating agent; a lubricant; a glidant; a sweetening agent; and a flavoring agent.

Examples of binders include microcrystalline cellulose, gum tragacanth, glucose solution, acacia mucilage, gelatin solution, sucrose, and starch paste. Lubricants include talc, starch, magnesium or calcium stearate, lycopodium and stearic acid. Diluents include, for example, lactose, sucrose, starch, kaolin, salt, mannitol, and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate, and polyoxyethylene laural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

If oral administration is desired, the compound can be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. Higher concentrations, up to about 98% by weight of the chemical entities described herein may be included.

Pharmaceutically acceptable carriers included in tablets are binders, lubricants, diluents, disintegrating agents, coloring agents, flavoring agents, and wetting agents. Enteric-coated tablets, because of the enteric-coating, resist the action of stomach acid and dissolve or disintegrate in the neutral or alkaline intestines. Sugar-coated tablets are compressed tablets to which different layers of pharmaceutically acceptable substances are applied. Film-coated tablets are compressed tablets which have been coated with a polymer or other suitable coating. Multiple compressed tablets are compressed tablets made by more than one compression cycle utilizing the pharmaceutically acceptable substances previously mentioned. Coloring agents may also be used in the above dosage forms. Flavoring and sweetening agents are used in compressed tablets, sugar-coated, multiple compressed and chewable tablets. Flavoring and sweetening agents may be useful in the formation of chewable tablets and lozenges.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents may be used in the above dosage forms.

Solvents include glycerin, sorbitol, ethyl alcohol and syrup. Examples of preservatives include glycerin, methyl and propylparaben, benzoic add, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Diluents include lactose and sucrose. Sweetening agents include sucrose, syrups, glycerin and artificial sweetening agents such as saccharin. Wetting agents include propylene glycol monostearate, sorbitan monooleate, diethylene glycol monolaurate, and polyoxyethylene lauryl ether. Organic acids include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, may be encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. Re 28,819 and 4,358,603. Briefly, such formulations include, but are not limited to, those containing a compound provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl)acetals of lower alkyl aldehydes such as acetaldehyde diethyl acetal.

In some embodiments, tablets and capsules formulations may be coated as known by those of skill in the art in order to modify or sustain dissolution of the chemical entities described herein. Thus, for example, they may be coated with a conventional enterically digestible coating, such as phenylsalicylate, waxes and cellulose acetate phthalate.

Parenteral administration, generally characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Implantation of a slow-release or sustained-release system, such that a constant level of dosage is maintained (see, e.g., U.S. Pat. No. 3,710,795) is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations may be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the patient or animal as is known in the art.

The unit-dose parenteral preparations are packaged in an ampoule, a vial or a syringe with a needle. Preparations for parenteral administration should be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. Typically a therapeutically effective dosage is formulated to contain a concentration of at least about 0.1% w/w up to about 90% w/w or more, such as more than 1% w/w of the active compound to the treated tissue(s). The chemical entities described herein may be administered at once, or may be divided into a number of smaller doses to be administered at intervals of time. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

The chemical entities described herein may be suspended in micronized or other suitable form or may be derivatized to produce a more soluble active product or to produce a prodrug. The form of the resulting mixture depends upon a number of factors, including the intended mode of administration and the solubility of the chemical entity in the selected carrier or vehicle. The effective concentration is sufficient for ameliorating the symptoms of the condition and may be empirically determined.

Of interest herein are also lyophilized powders, which can be reconstituted for administration as solutions, emulsions and other mixtures. They may also be reconstituted and formulated as solids or gels.

The sterile, lyophilized powder is prepared by dissolving at least one chemical entity described herein in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, typically, about neutral pH. Subsequent sterile filtration of the solution followed by lyophilization under standard conditions known to those of skill in the art provides the desired formulation. Generally, the resulting solution will be apportioned into vials for lyophilization. Each vial will contain a single dosage (10-1000 mg, such as 100-500 mg) or multiple dosages. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, such as 5-35 mg, for example, about 9-30 mg of lyophilized powder, is added per mL of sterile water or other suitable carrier. The precise amount depends upon the selected chemical entity. Such amount can be empirically determined.

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The chemical entities described herein may be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will typically have diameters of less than 50 microns, such as less than 10 microns.

The chemical entities described herein may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the chemical entity alone or in combination with other pharmaceutically acceptable excipients can also be administered.

These solutions, particularly those intended for ophthalmic use, may be formulated as 0.01%-10% isotonic solutions, pH about 5-7, with appropriate salts.

Other routes of administration, such as topical application, transdermal patches, and rectal administration are also contemplated herein.

Transdermal patches, including iontophoretic and electrophoretic devices, are well known to those of skill in the art. For example, such patches are disclosed in U.S. Pat. Nos. 6,267,983, 6,261,595, 6,256,533, 6,167,301, 6,024,975, 6,010,715, 5,985,317, 5,983,134, 5,948,433, and 5,860,957.

Pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The typical weight of a rectal suppository is about 2 to 3 gm.

Tablets and capsules for rectal administration are manufactured using the same pharmaceutically acceptable substance and by the same methods as for formulations for oral administration.

The chemical entities described herein may also be formulated to be targeted to a particular tissue, receptor, or other area of the body of the subject to be treated. Many such targeting methods are well known to those of skill in the art and are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In some embodiments, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a chemical entity described herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

The chemical entities described herein may be packaged as articles of manufacture containing packaging material, the chemical entity provided herein within the packaging material, and a label that indicates the uses for the chemical entity.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the chemical entities described herein are contemplated as are a variety of treatments for any disease or disorder in which nuclear receptor activity, including the farnesoid X receptor and/or orphan nuclear receptor activity, is implicated as a mediator or contributor to the symptoms or cause.

Starting materials and reagents in the synthesis examples provided herein are either available from commercial sources or via literature procedures (e.g., March Advanced Organic Chemistry: Reactions, Mechanisms, and Structure, (1992) 4th Ed.; Wiley Interscience, New York). Commercially available compounds generally were used without further purification unless otherwise indicated.

It is understood that in the following description, combinations of substituents and/or variables of the depicted formulae are permissible only if such contributions result in stable compounds under standard conditions.

One of ordinary skill in the art can easily ascertain which choices for each substituent are possible for the reaction conditions of each Scheme. Moreover, the substituents are selected from components as indicated in the specification heretofore, and may be attached to starting materials, intermediates, and/or final products according to schemes known to those of ordinary skill in the art.

Also it will be apparent that many of the products could exist as one or more isomers, that is E/Z isomers, enantiomers and/or diastereomers.

It will also be appreciated by those skilled in the art that in the process described below the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (e.g., t-butyldimethylsilyl, t-butyldiphenylsilyl or trimethylsilyl), tetrahydropyranyl, benzyl, and the like. Suitable protecting groups for amino include t-butoxycarbonyl, benzyloxycarbonyl, and the like. Suitable protecting groups for mercapto include —C(O)—R (where R is alkyl, aryl or aralkyl), p-methoxybenzyl, trityl and the like. Suitable protecting groups for carboxylic acid include alkyl, aryl or aralkyl esters.

Protecting groups may be added or removed in accordance with standard techniques, which are well-known to those skilled in the art and as described herein. The use of protecting groups is described in detail in Greene's Protective Groups in Organic Synthesis: (2006) 4th Ed., Wiley, John & Sons, Chapter 7, pp 696-926, which is incorporated herein by reference.

In some embodiments, compounds of formula I are produced by the following reaction schemes.

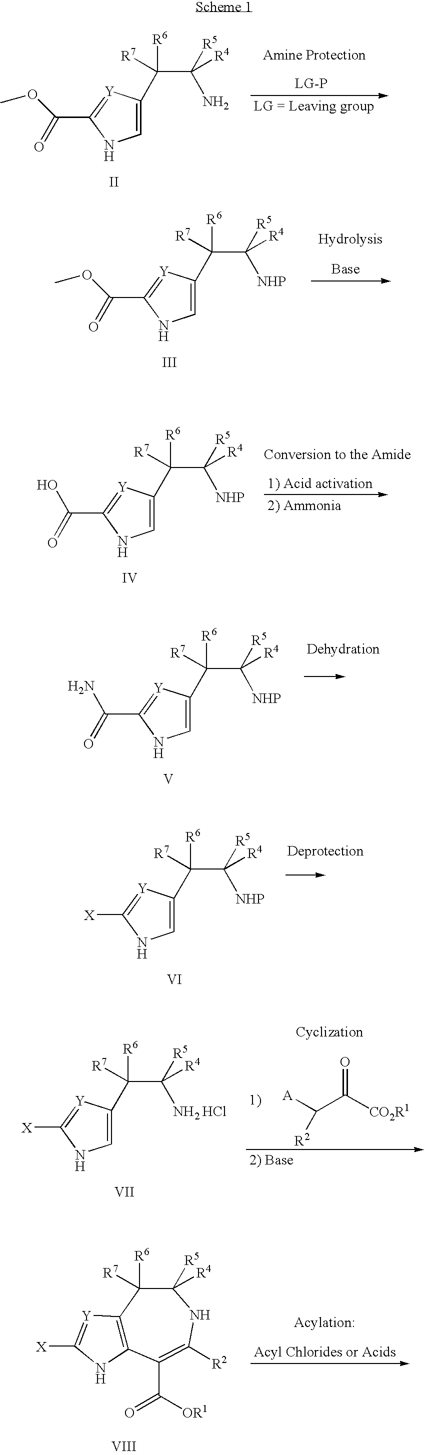

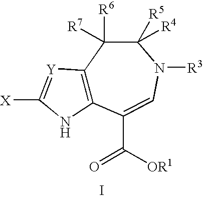

Compounds of formula I where Y is CR⁸ or N, X is CN, and R¹, R², R³, R⁴, R⁵, R⁶, R⁷, and R⁸ are as previously described can be prepared from compounds of formula VIII via acylation. The reaction is carried out by any conventional method for amine substitution. Acylation of the amine can be achieved by any conventional method for the formation of a peptide bond including but not limited to: 1) treatment of compounds of formula VIII with a carboxylic acid and a coupling agent including but not limited to: HATU, BOP, EDC/DMAP, and EDC/HOBt; 2) treatment of compounds of formula VIII with base and an acyl chloride. In some embodiments, the azepine nitrogen is treated with the appropriate acyl chloride in the presence of triethylamine.

Compounds of formula VIII can be prepared from compounds of formula VII via cyclization followed by a rearrangement reaction. Any conventional method to form the appropriate azepine ring can be employed. In some embodiments, compounds of formula VII are treated with either iso-propylbromopyruvate, iso-propylchloropyruvate, or a mixture of the two and heated (e.g., at 80° C.). Upon completion of the cyclization, pyridine and DMAP are added and heated (e.g., at 80° C.) to effect rearrangement.

Compounds of formula VII where X is CN can be prepared from compounds of formula VI via the removal of the amino protecting group (P is any conventional amine protecting group; for a review of suitable amine and pyrrole protecting groups and the use thereof see: Greene, T. W.; Wutts, P. G. M. Protective Groups in Organic Synthesis, 3rd ed.; Wiley and Sons: New York, 1999), followed by the preparation of a salt of the resulting amine. Where P is tert-butoxycarbonyl, any conventional method for the deprotection of a carbamate can be utilized. In some embodiments, compounds of formula VI are treated with 6 N HCl at room temperature to effect both transformations.

Compounds of formula VI can be prepared from compounds of formula IV by activation of the carboxylic acid, followed by displacement to form amides of formula V and dehydration to form the nitrile VI. Any conventional method for converting a carboxylic acid to an amide followed by any conventional method for converting an amide to a nitrile can be utilized. In some embodiments, carboxylic acids of formula IV are converted to the acid chloride using thionyl chloride and the acid chloride is treated with ammonia in ether to generate the corresponding amide V. Compounds of formula V are then treated with trifluoroacetic anhydride at room temperature to form the nitrile VI.

Compounds of formula IV are prepared from compounds of formula III via ester hydrolysis. The conversion of the ester to the carboxylic acid can be accomplished using any conventional method for the hydrolysis of an ester. In some embodiments, compounds of formula III are treated with an aqueous solution of 1N sodium hydroxide and heated (e.g., at 50° C.).

Compounds of formula III are prepared from compounds of formula II via protection of the amino group. The amino group can be masked using any conventional protecting group. In some embodiments, amines of formula II are treated with di-tert-butyldicarboxylate at room temperature.

Compounds of formula II are prepared as reported previously (see, e.g., WO2005009387).

The cyclization of compounds of formula VII to form an azepine ring system can be accomplished using the appropriate halopyruvate, as shown in Scheme 2, to yield compounds of formula VIII.

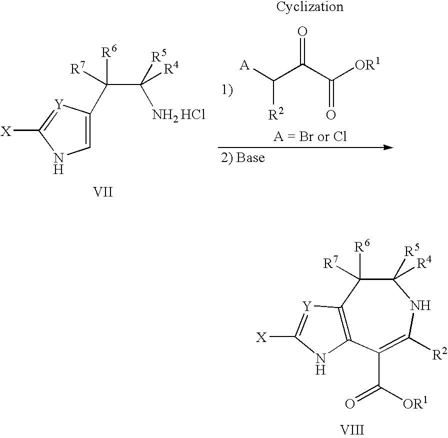

The reaction sequence can be carried out using any conventional Pictet-Spengler procedure, followed by treatment of the resulting halide with base. In some embodiments, compounds of formula VII are heated with halopyruvate (e.g., at 80° C.). Once cyclization is completed, rearrangement is effected by heating with pyridine and DMAP (e.g., at 80° C.).

Halopyruvate esters can be formed from the corresponding pyruvic acid. The conversion can be accomplished using any conventional esterification method. In some embodiments, bromopyruvic acid is treated with thionyl chloride and an alcohol of formula R¹OH.

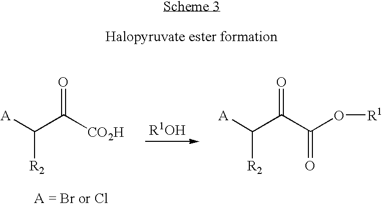

Halopyruvate esters can also be formed from an alcohol via oxidative bromination as depicted in Scheme 4. The conversion can be accomplished via any conventional method for the one-step oxidation and bromination of an alcohol. In some embodiments, the requisite alcohol is treated with bromine and acetic acid to yield the desired halopyruvate.

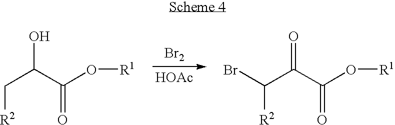

Compounds of formula IX can be prepared by the hydrolysis or cleavage of compounds of formula I as shown in Scheme 5. The conversion can be accomplished using any conventional method for hydrolysis or cleavage of an ester. In some embodiments, compounds of formula I and lithium chloride in DMF are either irradiated in a microwave (e.g., at 180° C.) or heated (e.g., at reflux).

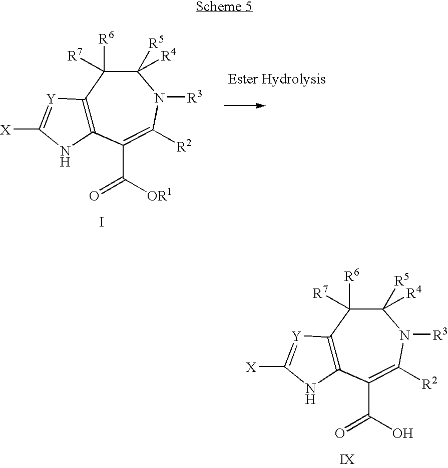

Amides of formula X can be prepared from carboxylic acids of formula IX as depicted in Scheme 6. The conversion can be performed using any conventional acid activating reagent including, but not limited to: HATU, BOP, EDC/DMAP, and EDC/HOBt and treatment with amine. In some embodiments, compounds of formula IX are treated with HATU and the requisite amine in NMP.

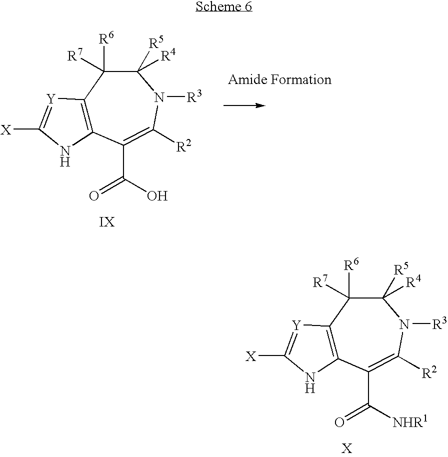

Spirocycles of formula XIII can be formed from compounds of formula XI via the reaction sequence shown in Scheme 7.

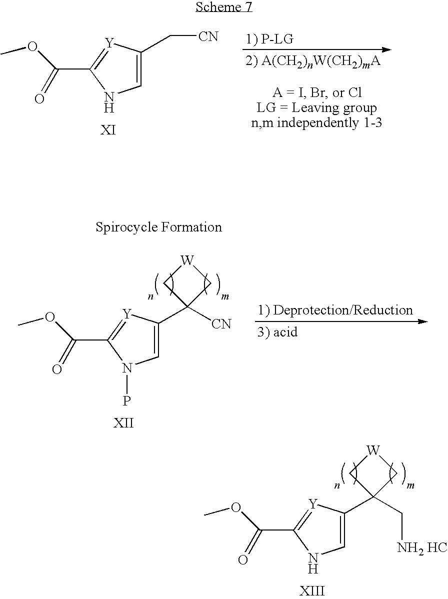

The pyrrole nitrogen of compounds of formula XI is protected with any suitable protecting group (P) and the compound is subsequently treated with a dihalo-alkylating agent where W is a heteroatom or methylene (both optionally substituted) to yield compounds of formula XII. Where W is nitrogen, a protecting group orthogonal to P can be used. In some embodiments, compounds of formula XI are treated with di-tert-butyldicarbonate and alkylated with di-bromopentane or bis(2-bromoethyl)ether. The protection and alkylation step can be achieved using any conventional method in the literature. The nitrile of formula XII is reduced, deprotected, and treated with an acid to yield the amine salt of formula XIII where HC is an acid counterion. Reduction of compounds of formula XII can be accomplished using any conventional method for the reduction of a nitrile including, but not limited to: lithium aluminum hydride, Raney nickel, and diisobutylaluminum hydride. In some embodiments, compounds of formula XI are treated with TFA to remove the protecting group, reduced with lithium aluminum hydride, and treated with HCl to form the amine salt of formula XIII.

The following examples are provided for illustrative purposes only and are not intended to limit the scope of the invention.

### EXAMPLES

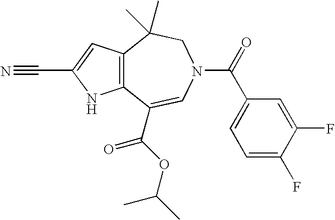

### Example 1

### iso-Propyl 2-cyano-6-(3,4-difluorobenzoyl)-4,4-dimethyl-1,4,5,6-tetrahydropyrrolo[2,3-d]azepine-8-carboxylate

Step 1: To a solution of methyl 4-(1-amino-2-methylpropan-2-yl)-1H-pyrrole-2-carboxylate (3.1 g, 15.5 mmol) in THF (65 mL) was added triethylamine (6.8 g, 46.5 mmol) and di-tert-butyl dicarbonate (6.4 g, 31.0 mmol). The reaction was stirred at room temperature for 30 min and concentrated to near dryness. The residue was partitioned between ethyl acetate and water, and the aqueous layer was extracted with ethyl acetate. The combined organic extracts were washed with water and brine, dried with magnesium sulfate, and concentrated. The crude product was purified by chromatography on silica gel (0-45% ethyl acetate/hexane) to yield the pure methyl 4-{2-[(tert-butoxycarbonyl)amino]-1,1-dimethylethyl}-1H-pyrrole-2-carboxylate. MS: (M+H-tBoc)⁺=197.

Step 2: A solution of methyl 4-{2-[(tert-butoxycarbonyl)amino]-1,1-dimethylethyl}-1H-pyrrole-2-carboxylate (3.3 g, 11.1 mmol) in acetonitrile (175 mL) and 1N aqueous solution of sodium hydroxide (55.7 mL) was heated at 70° C. for 3 h, stirred at room temperature for 18 h, then heated at 70° C. for 1 h. The reaction was cooled to 0° C., and ethyl acetate followed by a 1N aqueous solution of hydrochloric acid were added. The aqueous layer was extracted with ethyl acetate, and the combined organic extracts were washed with water and brine, dried with magnesium sulfate, and concentrated to yield the pure 4-{2-[(tert-Butoxycarbonyl)amino]-1,1-dimethylethyl}-1H-pyrrole-2-carboxylic acid. (M−H)⁻=281.

Step 3: To a solution of 4-{2-[(tert-butoxycarbonyl)amino]-1,1-dimethylethyl}-1H-pyrrole-2-carboxylic acid (435 mg, 1.54 mmol) in dichloromethane (20 mL) at 0° C. was added thionyl chloride (0.337 mL, 4.63 mmol). The reaction was allowed to warm to room temperature, stirred for 2 h, then concentrated to dryness to yield the crude acid chloride.

Ammonia gas was bubbled through diethyl ether (10 mL) for 5 min. This saturated diethyl ether solution was added to a solution of the crude acid chloride in diethyl ether (10 mL). After 15 minutes, the reaction was only 25% complete so ammonia gas was bubbled directly into the reaction mixture for 5 min. The reaction was capped, stirred for 18 h at room temperature, and concentrated to dryness, to yield the crude amide that was used directly in the next reaction.

To a mixture of the amide (˜460 mg, 1.64 mmol) in pyridine (25 mL) at 0° C. was added trifluoroacetic anhydride (0.912 mL, 6.56 mmol). Upon addition of the trifluoroacetic anhydride, the reaction turned yellow in color and gas evolution was observed. The reaction was allowed to warm to room temperature. After 15 min, additional trifluoroacetic anhydride (0.456 mL, 3.28 mmol) was added, and the reaction was stirred at room temperature for 18 h. The reaction was then concentrated and partitioned between ethyl acetate and a saturated aqueous sodium bicarbonate solution. The aqueous layer was then extracted with ethyl acetate, and the combined organic extracts were washed with brine, dried with magnesium sulfate, and concentrated. The crude product was purified by chromatography on silica gel (10-70% ethyl acetate/hexane) to yield the pure tert-butyl [2-(5-cyano-1H-pyrrol-3-yl)-2-methylpropyl]carbamate (210 mg, 52% from the carboxylic acid). MS: (M−H)⁻=262.

Step 4: To the protected amine (167 mg, 0.63 mmol) in acetonitrile (5 mL) was added 6 N HCl (5 mL). The reaction was stirred at room temperature for 18 h and then concentrated to dryness to yield pure 4-(2-amino-1,1-dimethylethyl)-1H-pyrrole-2-carbonitrile hydrochloride. MS: (M+H)⁺=164.

Step 5: To a mixture of 4-(2-amino-1,1-dimethylethyl)-1H-pyrrole-2-carbonitrile hydrochloride (370 mg, 2.3 mmol) in acetonitrile (8 mL) and isopropyl alcohol (8 mL) was added isopropylbromopyruvate (587 mg, 2.8 mmol). The reaction was heated at 80° C. for 18 h. The reaction was cooled to room temperature, and pyridine (0.518 mL, 6.4 mmol) and DMAP (17 mg, 0.138 mmol) were added. The reaction was heated at 80° C. for 18 h. The reaction was then concentrated and partitioned between dichloromethane and water. The aqueous layer was extracted with dichloromethane, and the combined organic extracts were washed with water and brine, dried with magnesium sulfate, and concentrated. The crude product was purified by chromatography on silica gel (10-45% ethyl acetate/hexane) to yield the pure iso-propyl 2-cyano-4,4-dimethyl-1,4,5,6-tetrahydropyrrolo[2,3-d]azepine-8-carboxylate. MS: (M+H)⁺=274.

Step 6: To a solution of iso-propyl 2-cyano-4,4-dimethyl-1,4,5,6-tetrahydropyrrolo[2,3-d]azepine-8-carboxylate (60 mg, 0.22 mmol) and triethylamine (46 μL, 0.33 mmol) in acetonitrile (1 mL) was added a solution of 3,4-difluorobenzoylchloride (41 μL, 0.33 mmol) in acetonitrile (0.1 mL). The reaction was stirred at room temperature for 18 h. The reaction was partitioned between ethyl acetate and water, and the aqueous layer was extracted with ethyl acetate. The combined organic extracts were then washed with water and brine, dried with magnesium sulfate, and concentrated. The crude product was purified by chromatography on silica gel (10-25% ethyl acetate/hexane) to yield the pure iso-propyl 2-cyano-6-(3,4-difluorobenzoyl)-4,4-dimethyl-1,4,5,6-tetrahydropyrrolo[2,3-d]azepine-8-carboxylate. MS: (M+H)⁺=414.

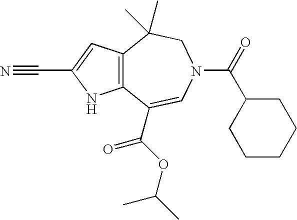

### Example 2

### iso-Propyl 2-cyano-6-(cyclohexylcarbonyl)-4,4-dimethyl-1,4,5,6-tetrahydropyrrolo[2,3-d]azepine-8-carboxylate

Prepared in an analogous manner to Example 1, step 6 from iso-propyl 2-cyano-4,4-dimethyl-1,4,5,6-tetrahydropyrrolo[2,3-d]azepine-8-carboxylate and cyclohexylcarbonyl chloride. MS (ES) m/z 382.1 ([M−H]⁻).

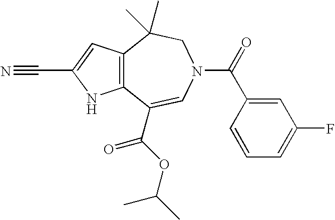

### Example 3

### iso-Propyl 2-cyano-6-(3-fluorobenzoyl)-4,4-dimethyl-1,4,5,6-tetrahydropyrrolo[2,3-d]azepine-8-carboxylate

Prepared in an analogous manner to Example 1, step 6 from iso-propyl 2-cyano-4,4-dimethyl-1,4,5,6-tetrahydropyrrolo[2,3-d]azepine-8-carboxylate and 3-fluorobenzoyl chloride. MS (ES) m/z 394.1 ([M−H]⁻).

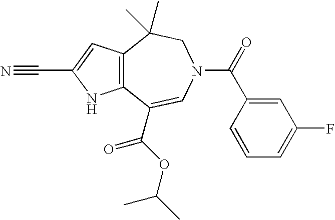

### Example 4

### iso-Propyl 2-cyano-6-(4-fluorobenzoyl)-4,4-dimethyl-1,4,5,6-tetrahydropyrrolo[2,3-d]azepine-8-carboxylate

Prepared in an analogous manner to Example 1, step 6 from iso-propyl 2-cyano-4,4-dimethyl-1,4,5,6-tetrahydropyrrolo[2,3-d]azepine-8-carboxylate and 4-fluorobenzoyl chloride. MS (ES) m/z 394.1 ([M−H]⁻).

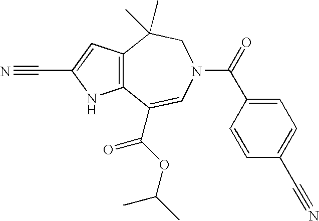

### Example 5

### iso-Propyl 2-cyano-6-(4-cyanobenzoyl)-4,4-dimethyl-1,4,5,6-tetrahydropyrrolo[2,3-d]azepine-8-carboxylate

Prepared in an analogous manner to Example 1, step 6 from iso-propyl 2-cyano-4,4-dimethyl-1,4,5,6-tetrahydropyrrolo[2,3-d]azepine-8-carboxylate and 4-cyanobenzoyl chloride. MS (ES) m/z 401.1 ([M−H]⁻).

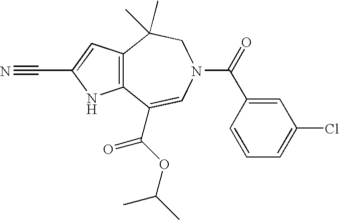

### Example 6

### iso-Propyl 6-(3-chlorobenzoyl)-2-cyano-4,4-dimethyl-1,4,5,6-tetrahydropyrrolo[2,3-d]azepine-8-carboxylate

Prepared in an analogous manner to Example 1, step 6 from iso-propyl 2-cyano-4,4-dimethyl-1,4,5,6-tetrahydropyrrolo[2,3-d]azepine-8-carboxylate and 3-chlorobenzoyl chloride. MS (ES) m/z 410.1/412.1 ([M−H]⁻).

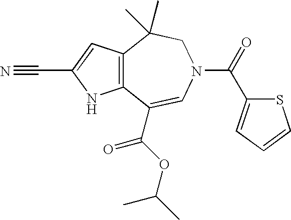

### Example 7

### iso-Propyl 2-cyano-4,4-dimethyl-6-(2-thienylcarbonyl)-1,4,5,6-tetrahydropyrrolo[2,3-d]azepine-8-carboxylate

Prepared in an analogous manner to Example 1, step 6 from iso-propyl 2-cyano-4,4-dimethyl-1,4,5,6-tetrahydropyrrolo[2,3-d]azepine-8-carboxylate and 2-thiophenecarbonyl chloride. MS (ES) m/z 382.1 ([M−H]⁻).

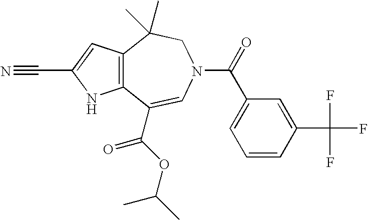

### Example 8

### iso-Propyl 2-cyano-4,4-dimethyl-6-[3-(trifluoromethyl)benzoyl]-1,4,5,6-tetrahydropyrrolo[2,3-d]azepine-8-carboxylate

Prepared in an analogous manner to Example 1, step 6 from iso-propyl 2-cyano-4,4-dimethyl-1,4,5,6-tetrahydropyrrolo[2,3-d]azepine-8-carboxylate and 3-(trifluoromethyl)benzoyl chloride. MS (ES) m/z 444.2 ([M−H]⁻).

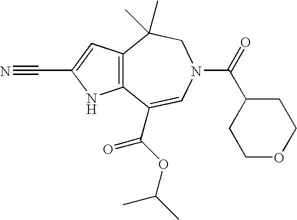

### Example 9

### iso-Propyl 2-cyano-4,4-dimethyl-6-(tetrahydro-2H-pyran-4-ylcarbonyl)-1,4,5,6-tetrahydropyrrolo[2,3-d]azepine-8-carboxylate

Prepared in an analogous manner to Example 1, step 6 from iso-propyl 2-cyano-4,4-dimethyl-1,4,5,6-tetrahydropyrrolo[2,3-d]azepine-8-carboxylate and tetrahydro-2H-pyran-4-carbonyl chloride. MS (ES) m/z 385.9 ([M+H]⁺).

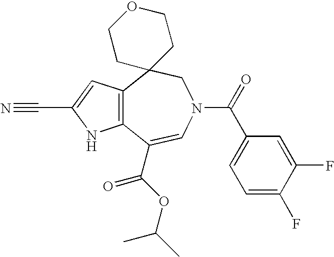

### Example 10

### iso-Propyl 2′-cyano-6′-(3,4-difluorobenzoyl)-2,3,5,5′,6,6′-hexahydro-1′H-spiro[pyran-4,4′-pyrrolo[2,3-d]azepine]-8′carboxylate

Step 1: To a solution of methyl 4-(cyanomethyl)-1H-pyrrole-2-carboxylate (470 mg, 2.87 mmol) in 50% aqueous NaOH (3.1 mL) was added Triton B (47 μL, 40% in methanol), and benzylbromide (0.69 mL, 5.73 mmol). The reaction was stirred at room temperature for 1 h. Water was added and the mixture was extracted with ethyl acetate (2×). The combined organic extracts were washed with brine (1×), dried over magnesium sulfate and concentrated. The crude material was purified by chromatography on silica gel (0-45% ethyl acetate/hexane) to yield the pure methyl 1-benzyl-4-(cyanomethyl)-1H-pyrrole-2-carboxylate. MS: (M+H)⁺=254.9.

Step 2: To a 1 L flask filled with 2-chloroethylether (7 mL, 60 mmol) and acetone (280 mL) was added sodium iodide (45 g, 300 mmol). The reaction was heated at reflux under nitrogen for 7 d. The reaction was filtered and the filtrate was concentrated to yield crude 1-iodo-2-(2-iodoethoxy)ethane.

To a mixture of 1-iodo-2-(2-iodoethoxy)ethane (848 mg, 2.6 mmol), LHMDS (5 mL, 1.0 M), and THF (5 mL) was added a solution of methyl 1-benzyl-4-(cyanomethyl)-1H-pyrrole-2-carboxylate (500 mg, 2.0 mmol) in THF (2.5 mL). The reaction was complete after stirring at room temperature for 15 min. An aqueous solution of saturated ammonium chloride was added followed by ethyl acetate and water. The mixture was extracted with ethyl acetate (1×) and the combined organic extracts were washed with brine (1×), dried over magnesium sulfate, and concentrated. The crude product was purified by chromatography on silica gel (0-50% ethyl acetate/hexane) to yield the pure methyl 1-benzyl-4-(4-cyanotetrahydro-2H-pyran-4-yl)-1H-pyrrole-2-carboxylate. MS: (M+H)⁺=324.8.

Step 3: A solution of methyl 1-benzyl-4-(4-cyanotetrahydro-2H-pyran-4-yl)-1H-pyrrole-2-carboxylate (3.1 g, 9.57 mmol) in methanol (45 mL), THF (45 mL), and ammonium hydroxide (9 mL) was flushed with nitrogen (3×). Raney Nickel was added (6 mL) and the reaction was flushed with nitrogen (3×). A hydrogen balloon was affixed to the reaction and the reaction was flushed with hydrogen (3×). The reaction was stirred at room temperature for 18 h at which time all of the nitrile had been converted to the amine. The mixture was filtered and the filtrate was concentrated to approximately 30 mL.

To this 30 mL solution of was added THF (70 mL), triethylamine (2 mL, 14.36 mmol), and di-tert-butyl dicarbonate (2.3 g, 10.53 mmol). The reaction was stirred at room temperature for 1 h, then concentrated. The reaction was partitioned between ethyl acetate and water and extracted with ethyl acetate (1×). The combined organic extracts were washed with water (1×) and brine (1×), dried over magnesium sulfate and concentrated. The crude product was purified by chromatography on silica gel (0-50% ethyl acetate/hexane) to yield pure methyl 1-benzyl-4-(4-{[(tert-butoxycarbonyl)amino]methyl}tetrahydro-2H-pyran-4-yl)-1H-pyrrole-2-carboxylate MS: (M+H)⁺=428.9.

Step 4: A 3-neck round bottom flask fitted with a condenser tube was placed under a nitrogen atmosphere and cooled to −78° C. Ammonia gas was bubbled into the flask until 50 mL of condensed ammonia accumulated. Sodium metal (1.2 g, 52 mmol)(prewashed with hexane) was added and the ammonia/sodium mixture turned dark blue. To this mixture was added a solution of methyl 1-benzyl-4-(4-{[(tert-butoxycarbonyl)amino]methyl}tetrahydro-2H-pyran-4-yl)-1H-pyrrole-2-carboxylate (3.3 g, 7.7 mmol) in THF (20 mL). The reaction turned yellow 1 min after addition of the pyrrole solution. The reaction was quenched with the careful addition of a saturated ammonium chloride solution and placed under a stream of nitrogen to evaporate the ammonia. The solution was extracted with DCM (2×); the combined organic extracts were washed with brine (1×), dried over magnesium sulfate, and concentrated. The crude 4-(4-((tert-butoxycarbonylamino)methyl)tetrahydro-2H-pyran-4-yl)-1H-pyrrole-2-carboxylic acid was used directly in the next step.

Step 5: tert-butyl (4-(5-cyano-1H-pyrrol-3-yl)tetrahydro-2H-pyran-4-yl)methylcarbamate was prepared in a manner analogous to Example 1, step 3 from 4-(4-((tert-butoxycarbonylamino)methyl)tetrahydro-2H-pyran-4-yl)-1H-pyrrole-2-carboxylic acid and used crude in the next step.

Step 6: 4-(4-(aminomethyl)tetrahydro-2H-pyran-4-yl)-1H-pyrrole-2-carbonitrile hydrochloride was prepared in a manner analogous to Example 1, step 4 from tert-butyl (4-(5-cyano-1H-pyrrol-3-yl)tetrahydro-2H-pyran-4-yl)methylcarbamate and used crude in the following step.

Step 7: (E)-iso-propyl 2′-cyano-2,3,5,5′,6,6′-hexahydro-1′H-spiro[pyran-4,4′-pyrrolo[2,3-d]azepine]-8′-carboxylate was prepared in a manner analogous to Example 1, step 5 from 4-(4-(aminomethyl)tetrahydro-2H-pyran-4-yl)-1H-pyrrole-2-carbonitrile hydrochloride. (M+H)⁺=315.7.

Step 8: Iso-propyl 2′-cyano-6′-(3,4-difluorobenzoyl)-2,3,5,5′,6,6′-hexahydro-1′H-spiro[pyran-4,4′-pyrrolo[2,3-d]azepine]-8′-carboxylate was prepared in a manner analogous to Example 1, step 6 from (E)-iso-propyl 2′-cyano-2,3,5,5′,6,6′-hexahydro-1′H-spiro[pyran-4,4′-pyrrolo[2,3-d]azepine]-8′-carboxylate. (M+H)⁺=455.7.

### Example 11

### Characterization of Compounds for Gal4/hFXR Fusion Protein Agonist Activity in Human 293 Cells

### Materials And Methods:

Assay Medium: Phenol red free high glucose Dulbecco's modified Eagle's medium with sodium pyruvate (Cellgro, #17-205-CV) supplemented with 10% fetal bovine serum (Gibco, 16000-044), 1% glutamax (Gibco, 35050-061), 100 units/mL penicillin and 100 μg/mL streptomycin (Gibco, 15140-122).
Culturplate-96 (PerkinElmer, 6005688)
Lysis buffer (Promega, E3971)
Luciferase assay reagent (Promega E1483)

### Procedure:

### Day 1.

1. Compounds to be tested are prepared as 2× stocks in assay medium.
2. Human 293 stable clone 2 expressing Gal4/hFXR fusion protein are thawed from frozen stock vials, added to 9 ml of assay medium, and centrifuged at 700 rpm in a Beckman Allegra 6R centrifuge for 10 minutes. The supernatant is removed and the cells are resuspended in 1 ml assay medium. The cells are counted and diluted in assay medium to 200,000 cells per ml. The cells are then plated at 10,000 cells per well in Culturplate-96 plates in 50 μL assay medium. The cells are incubated at 37° C. for approximately 1 hour.
3. 50 μl of 2× compounds in assay medium at 37° C. is added to each well. All assays include 1 uM GW4064 as a reference standard.
4. Cells are incubated for 24 hours at 37° C.

### Day 2.

5. The medium is removed, and the cells are lysed in 25 uL lysis buffer (Promega, E3971).
6. The plates are analyzed for luciferase activity with luciferase assay reagent (Promega E1483). Plates are read on Victor³V instrument using the protocol “Shuguang Luciferase assay” (dispense volume=100 uL, plate type=“Packard Viewplate”, measurement height=8 mm from bottom of plate, 5 second read per well).

### Analysis Of Results:

1. For agonist single point screening, data are analyzed in Excel. Each compound is tested in triplicate. The fold stimulation of each compound is calculated as RLU_{cpd}/RLU_{bkgd}.
2. Comparison to GW4064 is made by the equation (RLU_{cpd}/RLU_{bkgd})/(RLU_{GW4064}/RLU_{bkgd})
3. For agonist potency determinations, statistical analysis of the data is performed using a customized Excel/SAS program. Dose response curves are generated using a four parameter (min, max, slope, and EC₅₀ where EC₅₀ is defined as the concentration which corresponds to midway between the estimated max and min) logistic model using log-transformed data (data is transformed on both sides with known lambda=0).

| | hFXR EC₅₀ |
|---|---|
| Compound | (nM) |
| iso-Propyl 2-cyano-6-(3,4-difluorobenzoyl)-4,4-dimethyl-1,4,5,6- | 5.3 |
| tetrahydropyrrolo[2,3-d]azepine-8-carboxylate | |
| iso-Propyl 2-cyano-6-(cyclohexylcarbonyl)-4,4-dimethyl-1,4,5,6- | 7.0 |
| tetrahydropyrrolo[2,3-d]azepine-8-carboxylate | |
| iso-Propyl 2-cyano-6-(3-fluorobenzoyl)-4,4-dimethyl-1,4,5,6- | 3.0 |
| tetrahydropyrrolo[2,3-d]azepine-8-carboxylate | |
| iso-Propyl 2-cyano-6-(4-fluorobenzoyl)-4,4-dimethyl-1,4,5,6- | 5.0 |
| tetrahydropyrrolo[2,3-d]azepine-8-carboxylate | |
| iso-Propyl 2-cyano-6-(4-cyanobenzoyl)-4,4-dimethyl-1,4,5,6- | 42 |
| tetrahydropyrrolo[2,3-d]azepine-8-carboxylate | |
| iso-Propyl 6-(3-chlorobenzoyl)-2-cyano-4,4-dimethyl-1,4,5,6- | 13 |
| tetrahydropyrrolo[2,3-d]azepine-8-carboxylate | |
| iso-Propyl 2-cyano-4,4-dimethyl-6-(2-thienylcarbonyl)-1,4,5,6- | 22 |
| tetrahydropyrrolo[2,3-d]azepine-8-carboxylate | |
| iso-Propyl 2-cyano-4,4-dimethyl-6-[3-(trifluoromethyl)benzoyl]-1,4,5,6- | 8.0 |
| tetrahydropyrrolo[2,3-d]azepine-8-carboxylate | |
| iso-Propyl 2-cyano-4,4-dimethyl-6-(tetrahydro-2H-pyran-4-ylcarbonyl)- | 8.9 |
| 1,4,5,6-tetrahydropyrrolo[2,3-d]azepine-8-carboxylate | |
| iso-Propyl 2′-cyano-6′-(3,4-difluorobenzoyl)-2,3,5,5′,6,6′-hexahydro-1′H- | 86 |
| spiro[pyran-4,4′-pyrrolo[2,3-d]azepine]-8′carboxylate | |

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. At least one chemical entity chosen from compounds of Formula I 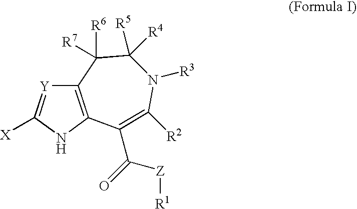
and pharmaceutically acceptable salts thereof, wherein
X is chosen from CN, CF₃, CF₂H, S(O)ₙR⁸, and S(O)₂N(R⁹)R¹⁰;
n is 0, 1, or 2;
Y is chosen from CR¹¹ and N;
Z is chosen from O and NH;
R¹ is chosen from optionally substituted alkyl, optionally substituted cycloalkyl, optionally substituted heterocyclyl, optionally substituted aryl, and optionally substituted heteroaryl;
R² is chosen from hydrogen and optionally substituted alkyl;
R³ is chosen from —C(O)R¹² and —C(O)N(R⁹)R¹⁰;
R⁴, R⁵, R⁶ and R⁷ are independently chosen from hydrogen and optionally substituted alkyl, or any two of R⁴, R⁵, R⁶ and R⁷, together with the atoms to which they are attached, form an optionally substituted cycloalkyl or optionally substituted heterocyclyl ring;
R⁸ is chosen from optionally substituted alkyl optionally substituted cycloalkyl, optionally substituted heterocyclyl, optionally substituted aryl, and optionally substituted heteroaryl;
each instance of R⁹ and R¹⁰ is independently chosen from hydrogen, optionally substituted aryl, optionally substituted heteroaryl, optionally substituted alkyl optionally substituted cycloalkyl, and optionally substituted heterocyclyl, or R⁹ and R¹⁰, together with the atoms to which they are attached, form an optionally substituted heterocyclyl ring;
R¹¹ is chosen from hydrogen and lower alkyl; and
R¹² is chosen from hydrogen, optionally substituted aryl, optionally substituted heteroaryl, optionally substituted alkyl optionally substituted cycloalkyl, and optionally substituted heterocyclyl.

2-3. (canceled)

4. At least one chemical entity of claim 1 wherein R⁴ and R⁵ are independently chosen from hydrogen and lower alkyl.

5. (canceled)

6. At least one chemical entity of claim 4 wherein R⁴ and R⁵ are hydrogen.

7-8. (canceled)

9. At least one chemical entity of claim 1 wherein Y is CR¹¹.

10. (canceled)

11. At least one chemical entity of claim 9 wherein R¹¹ is hydrogen.

12. (canceled)

13. At least one chemical entity of claim 1 wherein X is CN.

14-16. (canceled)

17. At least one chemical entity of claim 1 wherein R⁶ and R⁷ are independently chosen from hydrogen and lower alkyl.

18-20. (canceled)

21. At least one chemical entity of claim 17 wherein R⁶ and R⁷ are methyl.

22-23. (canceled)

24. At least one chemical entity of claim 1 wherein R¹ is lower alkyl.

25. (canceled)

26. At least one chemical entity of claim 24 wherein R¹ is iso-propyl.

27. (canceled)

28. At least one chemical entity of claim 1 wherein R² is chosen from hydrogen and lower alkyl.

29. At least one chemical entity of claim 28 wherein R² is hydrogen.

30. At least one chemical entity of claim 1 wherein R³ is —C(O)R¹².

31. (canceled)

32. At least one chemical entity of claim 30 wherein R¹² is chosen from cycloalkyl, heterocyclyl, phenyl, and heteroaryl, each of which is optionally substituted with one, two or three groups independently chosen from halo, cyano, lower alkyl, lower alkyl substituted with one, two, or three halo groups, hydroxy, and lower alkoxy.

33. At least one chemical entity of claim 32 wherein R¹² is chosen from cyclohexyl, phenyl, and thienyl, each of which is optionally substituted with one, two or three groups independently chosen from halo, cyano, lower alkyl, lower alkyl substituted with one, two, or three halo groups, hydroxy, and lower alkoxy.

34. At least one chemical entity of claim 33 wherein R¹² is chosen from hydrogen, 3,4-difluorophenyl, cyclohexyl, 3-fluorophenyl, 4-fluorophenyl, 4-cyanophenyl, 3-chlorophenyl, thienyl, and 3-trifluorophenyl.

35-38. (canceled)

39. At least one chemical entity of claim 1 wherein Z is O.

40. (canceled)

41. At least one chemical entity of claim 1 wherein the compound of Formula I is chosen from
Isopropyl 2-cyano-6-(3,4-difluorobenzoyl)-4,4-dimethyl-1,4,5,6-tetrahydropyrrolo[2,3-d]azepine-8-carboxylate;
Isopropyl 2-cyano-6-(cyclohexylcarbonyl)-4,4-dimethyl-1,4,5,6-tetrahydropyrrolo[2,3-d]azepine-8-carboxylate;
Isopropyl 2-cyano-6-(3-fluorobenzoyl)-4,4-dimethyl-1,4,5,6-tetrahydropyrrolo[2,3-d]azepine-8-carboxylate;
Isopropyl 2-cyano-6-(4-fluorobenzoyl)-4,4-dimethyl-1,4,5,6-tetrahydropyrrolo[2,3-d]azepine-8-carboxylate;
Isopropyl 2-cyano-6-(4-cyanobenzoyl)-4,4-dimethyl-1,4,5,6-tetrahydropyrrolo[2,3-d]azepine-8-carboxylate;
Isopropyl 6-(3-chlorobenzoyl)-2-cyano-4,4-dimethyl-1,4,5,6-tetrahydropyrrolo[2,3-d]azepine-8-carboxylate;
Isopropyl 2-cyano-4,4-dimethyl-6-(2-thienylcarbonyl)-1,4,5,6-tetrahydropyrrolo[2,3-d]azepine-8-carboxylate;
Isopropyl 2-cyano-4,4-dimethyl-6-[3-(trifluoromethyl)benzoyl]-1,4,5,6-tetrahydropyrrolo[2,3-d]azepine-8-carboxylate;
Isopropyl 2-cyano-4,4-dimethyl-6-(tetrahydro-2H-pyran-4-ylcarbonyl)-1,4,5,6-tetrahydropyrrolo[2,3-d]azepine-8-carboxylate; and
Isopropyl 2′-cyano-6′-(3,4-difluorobenzoyl)-2,3,5,5′,6,6′-hexahydro-1′H-spiro[pyran-4,4′-pyrrolo[2,3-d]azepine]-8′carboxylate.

42. A pharmaceutical composition comprising a pharmaceutically acceptable carrier or excipient and at least one chemical entity of claim 1.

43. (canceled)

44. A method of treating or preventing one or more symptoms of a disease or disorder in which farnesoid X receptor activity is implicated, comprising administering to a subject in need thereof an effective amount of at least one chemical entity of claims 1, wherein the disease or disorder is selected from hyperlipidemia, hypercholesterolemia, hyperlipoproteinemia, hypertriglyceridemia, dyslipidemia, lipodystrophy, gallstone disease, atherosclerosis, atherosclerotic disease, atherosclerotic disease events, atherosclerotic cardiovascular disease, Syndrome X, diabetes mellitus, type II diabetes, insulin insensitivity, hyperglycemia, cholestasis, obesity, gallstone disease, acne vulgaris, acneiform skin conditions, Parkinson's disease, cancer, Alzheimer's disease, inflammation, immunological disorders, lipid disorders, obesity, conditions characterized by a perturbed epidermal barrier function, peripheral occlusive disease, ischemic stroke, conditions of disturbed differentiation or excess proliferation of the epidermis or mucous membrane, cardiovascular disorders, diabetic nephropathy, metabolic acidosis, hypertension, myocardial infarction, hypertension, heart failure, sepsis, osteoarthritis, rheumatoid arthritis, nonalcoholic fatty liver disease.

45-65. (canceled)

66. A method of reducing plasma cholesterol levels, in a subject in need thereof, comprising administering an effective amount of at least one chemical entity of claim 1.

67. A method of reducing plasma triglyceride levels in a subject in need thereof, comprising administering an effective amount of at least one chemical entity of claim 1.

68-70. (canceled)

71. At least one chemical entity of claim 1 wherein
X is chosen from CN, CF₃, and CF₂H;
R¹¹ is chosen from hydrogen and methyl;
R¹ is optionally substituted alkyl;
R¹² is chosen from cycloalkyl, heterocyclyl, phenyl, and heteroaryl, each of which is optionally substituted with one, two or three groups independently chosen from halo, cyano, lower alkyl, lower alkyl substituted with one, two, or three halo groups, hydroxy, and lower alkoxy;
R⁹ and R¹⁰ is independently chosen from hydrogen and optionally substituted alkyl;
R⁴ and R⁵ are independently chosen from hydrogen and optionally substituted alkyl; and
R⁶ and R⁷ are independently chosen from hydrogen and optionally substituted alkyl.
